(19) United States
(12) Patent Application Publication
(10) Pub. No.: US 2016/0146788 A1
(43) Pub. Date: May 26, 2016

(54) METHOD FOR SCREENING DRUGS FOR TREATING/PREVENTING MYELODYSPLASTIC SYNDROME, ETC.

(71) Applicant: KYOTO UNIVERSITY, Kyoto (JP)

(72) Inventors: Shinya Yamanaka, Kyoto-shi, Kyoto (JP); Yoshinori Yoshida, Kyoto-shi, Kyoto (JP); Kazuhisa Chonabayashi, Kyoto-shi, Kyoto (JP)

(21) Appl. No.: 14/893,470

(22) PCT Filed: May 23, 2014
(86) PCT No.: PCT/JP2014/063753

§ 371 Date: Nov. 23, 2015

(30) Foreign Application Priority Data
May 23, 2013 (JP) 2013-109385

(51) Int. Cl. G01N 33/50; A61K 35/18; A61K 35/19; A61K 35/15; C12N 5/078; C12N 5/0787

(52) U.S. Cl. CPC G01N 33/5073; C12N 5/0641; C12N 5/0642; C12N 5/0644; G01N 33/502; A61K 35/19; A61K 35/15; A61K 35/18; C12N 2506/45; C12N 2501/165; C12N 2501/2306; C12N 2501/2303; C12N 2501/2311; C12N 2501/125; C12N 2501/26; C12N 2501/14; C12N 2501/145; C12N 2501/22; C12N 2501/115; C12N 2501/155; C12N 2503/00; G01N 2500/10

(57) ABSTRACT

The present invention provides a method of screening for a therapeutic or prophylactic drug for acute myeloid leukemia or myelodysplastic syndrome, comprising the following steps:
(a) a step of forming colonies of hematopoietic progenitor cells induced from pluripotent stem (iPS) cells produced from non-T cells in blood mononuclear cells isolated from myelodysplastic syndrome patients, in the presence of a test substance and in the absence of the test substance, and
(b) a step of selecting the test substance as a candidate for a therapeutic or prophylactic drug for acute myeloid leukemia or myelodysplastic syndrome and the like when the colony number in the presence of the test substance increases from the colony number in the absence of the test substance.

### TECHNICAL FIELD

The present invention relates to a method of screening for a therapeutic and/or prophylactic drug for acute myeloid leukemia or myelodysplastic syndrome (MDS) which is a disease related thereto. The present invention also relates to a therapeutic agent for myelodysplastic syndrome (MDS) containing hematopoietic progenitor cells induced from normal iPS cells.

### BACKGROUND ART

Myelodysplastic syndrome (MDS) is a clonal, acquired hematopoietic disorder, and is a bone marrow disease with poor prognosis concurrently accompanied by treatment-resistant anemia that progresses chronically, and a preleukemic state that easily transits to blood cell decrease (refractory anemia) and acute myeloid leukemia. It is often found in relatively elderly people, and characteristically, the probability of developing the disease is several to several dozen times higher when chemotherapy or radiation therapy for malignant tumor is involved (secondary MDS). Combined with the aging society and spreading aggressive chemotherapy in recent years, MDS cases have been steadily increasing. There are only a few effective treatment methods among those available at present, and a treatment method with the possibility of permanent cure is only the allogeneic hematopoietic stem cell transplantation. However, the application thereof is limited to young people, and symptomatic therapy such as transfusion and the like is mainly performed for a large majority of aged patients, and most of them die of infections and MDS that turned into leukemia within several years. Model mouse is hardly available for MDS, and MDS cell cannot be engrafted even in immunodeficient mouse. Therefore, the development of a tool capable of reproducing and analyzing the pathology of MDS, which makes it possible to develop future treatment methods, and the development of a novel therapeutic drug or diagnostic drug have been earnestly desired.

On the other hand, in the field of regenerative medicine and the like, a technique for transdifferentiation of a cell convenient as a biomaterial into a cell type of interest is desired, and induced pluripotent stem cells (iPS cell) of mouse and human have been established one after another. Yamanaka et al. succeeded in establishing an iPS cell by introduction of 4 genes of Oct3/4, Sox2, Klf4 and c-Myc into human skin-derived fibroblasts (patent document 1 and non-patent document 1). The thus-obtained iPS cell, after preparation using the cells derived from a patient to be the treatment target, can be differentiated into the cell of each tissue, and therefore, the pathology is considered to be reproducible in vitro. However, there is no report, up to the present, on successful preparation of an iPS cell from a somatic cell derived from MDS patient.

### DOCUMENT LIST

### Patent Document

Patent document 1: W02007/069666

### Non-Patent Document

Non-patent document 1: Takahashi, K, et al., Cell. 131:861-872 (2007)

### SUMMARY OF THE INVENTION

### Problems to be Solved by the Invention

The problem of the present invention is to provide a method of screening for a therapeutic or prophylactic drug for acute myeloid leukemia or myelodysplastic syndrome (MDS) which is a disease related thereto. The problem of the present invention is to also provide a therapeutic agent for myelodysplastic syndrome (MDS).

### Means of Solving the Problems

The present inventors have conducted intensive studies in an attempt to solve the aforementioned problems and succeeded in reproducing the pathology of myelodysplastic syndrome (MDS) in the cells induced to differentiate from iPS cells derived from somatic cells of a myelodysplastic syndrome (MDS) patient. That is, it was found that hematopoietic progenitor cells induced to differentiate from iPS cells derived from non-T cells in blood mononuclear cells of an MDS patient show low colony-forming ability, and particularly poor in the differentiation potency into neutrophil, red blood cell and megakaryocyte. On the other hand, it was also revealed that, even in blood mononuclear cells of the same patient, hematopoietic progenitor cells induced to differentiate from T cell-derived iPS cells surprisingly does not show chromosome abnormality unique to MDS, and have normal colony-forming ability and differentiation potency into blood cells. The present invention has been completed based on such findings.

That is, the present invention provides the following matters.
[1] A method of screening for a therapeutic or prophylactic drug for acute myeloid leukemia or myelodysplastic syndrome, comprising the following steps:
(a) a step of forming colonies of hematopoietic progenitor cells induced from pluripotent stem (iPS) cells produced from non-T cells in blood mononuclear cells isolated from a myelodysplastic syndrome patient, in the presence of a test substance and in the absence of the test substance, and
(b) a step of selecting the test substance as a candidate for a therapeutic or prophylactic drug for acute myeloid leukemia or myelodysplastic syndrome when the colony number in the presence of the test substance increases from the colony number in the absence of the test substance.
[2] The method of [1] or [2], wherein the iPS cells produced from non-T cells in blood mononuclear cells isolated from the aforementioned myelodysplastic syndrome patient have at least one chromosome abnormality selected from the group consisting of increase in the copy number of short arm of chromosome 9 (9p), decrease in the copy number of short arm of chromosome 18 (18p), decrease in the copy number of long arm of chromosome 20 (20q), decrease in the copy number of long arm of chromosome 5 (5q), decrease in the copy number of long arm of chromosome 7 (7q), decrease in the copy number of long arm of chromosome 11 (11q), and translocation between long arm of chromosome 3 and long arm of chromosome 21.
[3] A method of screening for a therapeutic or prophylactic drug for acute myeloid leukemia or myelodysplastic syndrome, comprising the following steps:
(a) a step of inducing iPS cells produced from non-T cells in blood mononuclear cells isolated from a myelodysplastic syndrome patient into hematopoietic progenitor cells,
(b) a step of inducing the aforementioned hematopoietic progenitor cells into hematopoietic cells in the presence of a test substance, and in the absence of the test substance, and
(c) a step of selecting the test substance as a candidate for a therapeutic or prophylactic drug for acute myeloid leukemia or myelodysplastic syndrome when the number of hematopoietic cells in the presence of the test substance increases from the number of hematopoietic cells in the absence of the test substance.
[4] The method of [3], wherein the iPS cells produced from non-T cells in blood mononuclear cells isolated from the aforementioned myelodysplastic syndrome patient have at least one chromosome abnormality selected from the group consisting of increase in the copy number of short arm of chromosome 9 (9p), decrease in the copy number of short arm of chromosome 18 (18p), decrease in the copy number of long arm of chromosome 20 (20q), decrease in the copy number of long arm of chromosome 5 (5q), decrease in the copy number of long arm of chromosome 7 (7q), decrease in the copy number of long arm of chromosome 11 (11q), and translocation between long arm of chromosome 3 and long arm of chromosome 21.
[5] The method of [3] or [4], wherein the hematopoietic cells induced in the aforementioned step (b) are red blood cells.
[6] The method of [3] or [4], wherein the hematopoietic cells induced in the aforementioned step (b) are neutrophils.
[7] The method of [3] or [4], wherein the hematopoietic cells induced in the aforementioned step (b) are megakaryocytes.
[8] The method of [5], wherein the aforementioned step (b) comprises the following steps:
(i) a step of culturing hematopoietic progenitor cells in a is medium containing VEGF, IL-6, IL-3, IL-11, SCF, FLT3L, erythropoietin (EPO) and thrombopoietin (TPO),
(ii) a step of culturing the cells obtained in step (i) in a medium containing IL-3, SCF and EPO, and
(iii) a step of culturing the cells obtained in step (ii) in a medium containing SCF and EPO.
[9] The method of [6], wherein the aforementioned step (b) comprises a step of culturing the hematopoietic progenitor cells in a medium containing GM-CSF and/or G-CSF.
[10] The method of [7], wherein the aforementioned step (b) comprises a step of culturing the hematopoietic progenitor cells in a medium containing TPO and SCF.
[11] The method of any one of [3] to [10], wherein the aforementioned step (a) consists of the following steps:
(1) a step of forming embryoid bodies from iPS cells in a medium containing BMP-4,
(2) a step of culturing the aforementioned embryoid bodies in a medium containing bFGF and BMP-4, and
(3) a step of culturing the cells obtained in step (2) in a medium containing bFGF, VEGF, IL-6, IL-3, IL-11, stem cell factor (SCF) and FLT3L.
[12] The method of any of [3] to [10], wherein the aforementioned hematopoietic progenitor cells are hematopoietic progenitor cells induced by a method comprising a step of coculturing iPS cells with feeder cells.
[13] The method of [12], wherein the aforementioned feeder cell is OP9 cell line.
[14] A therapeutic agent for myelodysplastic syndrome, comprising hematopoietic progenitor cells prepared by differentiation induction of iPS cells produced from T cells of a myelodysplastic syndrome patient.
[15] The agent of [14], wherein the differentiation induction of the aforementioned iPS cells into hematopoietic progenitor cells consists of the following steps:
(1) a step of forming embryoid bodies from iPS cells in a medium containing BMP-4,
(2) a step of culturing the aforementioned embryoid bodies in a medium containing bFGF and BMP-4, and
(3) a step of culturing the cells obtained in step (2) in a medium containing bFGF, VEGF, IL-6, IL-3, IL-11, stem cell factor (SCF) and FLT3L.
[16] The agent of [14], wherein the aforementioned hematopoietic progenitor cells are hematopoietic progenitor cells induced by a method comprising a step of coculturing iPS cells with feeder cells.
[17] The agent of [16], wherein the aforementioned feeder cell is OP9 cell line.
[18] A method of screening for a therapeutic or prophylactic drug for acute myeloid leukemia or myelodysplastic syndrome, comprising the following steps:
(a) a step of contacting hematopoietic progenitor cells induced from induced pluripotent stem (iPS) cells produced from non-T cells in blood mononuclear cells isolated from a myelodysplastic syndrome patient (non-T cell-derived hematopoietic progenitor cells) and hematopoietic progenitor cells induced from control iPS cells (control hematopoietic progenitor cells) with a test substance, and
(b) a step of selecting the test substance as a candidate for a therapeutic or prophylactic drug for acute myeloid leukemia or myelodysplastic syndrome when the number of non-T cell-derived hematopoietic progenitor cells after contact with the test substance decreases from the number of control hematopoietic progenitor cells after contact with the test substance.
[19] The method of [18], wherein the aforementioned control hematopoietic progenitor cells are hematopoietic progenitor cells induced from iPS cells produced from T cells in blood mononuclear cells isolated from the same patient as non-T cells.
[20] The method of [18] or [19], wherein the iPS cells produced from non-T cells in blood mononuclear cells isolated from the aforementioned myelodysplastic syndrome patient have at least one chromosome abnormality selected from the group consisting of increase in the copy number of short arm of chromosome 9 (9p), decrease in the copy number of short arm of chromosome 18 (18p), decrease in the copy number of long arm of chromosome 20 (20q), decrease in the copy number of long arm of chromosome 5 (5q), decrease in the copy number of long arm of chromosome 7 (7q), decrease in the copy number of long arm of chromosome 11 (11q), and translocation between long arm of chromosome 3 and long arm of chromosome 21.
[21] The method of any of [18] to [20], wherein the aforementioned hematopoietic progenitor cells are hematopoietic progenitor cells induced from iPS cells by a method comprising the following steps:
(1) a step of culturing iPS cells in a medium containing BMP-4 to form embryoid bodies,
(2) a step of culturing the aforementioned embryoid bodies in a medium containing bFGF and BMP-4, and
(3) a step of culturing the cells obtained in step (2) in a medium containing bFGF, VEGF, IL-6, IL-3, IL-11, stem cell factor (SCF) and FLT3L.
[22] The method of any of [18] to [20], wherein the aforementioned hematopoietic progenitor cells are hematopoietic progenitor cells induced by a method comprising a step of coculturing iPS cells with feeder cells.
[23] The method of [22], wherein the aforementioned feeder cell is OP9 cell line.

### EFFECT OF THE INVENTION

According to the present invention, screening of a therapeutic and/or prophylactic drug for acute myeloid leukemia or myelodysplastic syndrome (MDS) is possible by using a novel tool. Particularly, since a cell derived from the same blood mononuclear cell from the same patient can be used as a control, conditions of the cell other than the presence or absence of pathology can be arranged, and a screening system with higher accuracy can be provided. The present invention enables provision of a therapeutic agent for myelodysplastic syndrome (MDS), which uses hematopoietic progenitor cells derived from somatic cells of a myelodysplastic syndrome patient. Therefore, the present invention is extremely useful for the treatment or prophylaxis of acute myeloid leukemia or myelodysplastic syndrome (MDS).

### BRIEF DESCRIPTION OF THE DRAWINGS

FIG. 1 shows stained images of the blood of MDS patients from which iPS cells are established. (A) shows a blast, bone marrow cell and giant platelet of KM3 (MDS patient), and (B) shows a blast, bone marrow cell, erythroblast cell, and megakaryocyte of KM5 (MDS patient).

FIG. 2 shows the measurement results of the copy number of genes (USP14, NDC810 and MYL12A) of chromosome 18p11.3 in iPS cells derived from the blood (non-T cells and T cells) of KM3.

FIG. 3 shows the karyotype and the results of CGH analysis of MDS-iPS cell. (A) shows the karyotype and the results of CGH analysis of KM3-A4 iPS cell line, and (B) shows one example of the karyotype of iPS cell line derived from KM5 and the results of CGH analysis of each iPS cell line.

FIG. 4 shows the results of KM5-derived iPS cell as analyzed by FISH in the interphase nucleus. Deficiency of chromosome 20q was confirmed (MDS-iPSCs).

FIG. 5 shows the results of RT-PCR analysis of pluripotent cell marker in each cell line. Mutant iPS cells (MDS-iPS) from MDS patients were confirmed to show equivalent expression of pluripotent cell marker as ES cells and the like.

FIG. 6 shows fluorescence images after immunostaining of pluripotent marker proteins (Tra-1-60 and SSEA4) of each iPS cell line (KM3-A4 and KM5-B3).

FIG. 7 shows photographs of teratoma produced from KM3-A4 and stained images of the sections thereof.

FIG. 8 shows a dendrogram of unsupervised hierarchical clustering based on global gene expression data.

FIG. 9 shows the results of differentiation induction of KM5-derived iPS cells into hematopoietic progenitor cells. (A) shows differentiation induced from normal iPS cells (T-iPS cell) established from KM5-derived T cells into hematopoietic progenitor cells, and (B) shows the results of flow cytometry on differentiation of KM5-derived mutant iPS cells (MDS-iPS cell) into hematopoietic progenitor cells. T-iPS cells or MDS-iPS cells were cultured on OP9 to allow for differentiation into hematopoietic progenitor cells.

FIG. 10 shows the results of colony formation unit (CFU) assay of hematopoietic progenitor cells (HPC) induced to differentiate from KM5-derived abnormal MDS-iPS cells (NonT-iPS in (A), MDS-iPSC in (B)) and normal MDS-iPS cells (T-iPS in (A), Normal iPSC in (B)). (A) shows the colony number per unit cells and the ratio of constituent components (BFU-E: red blood cell, GEMM: mixture, GM: granulocyte, macrophage, G: granulocyte, M: macrophage) in the colony, and (B) shows stained images of the colonies on day 15. 5M-B1 in (A) is an iPS cell line showing a normal karyotype.

FIG. 11 shows the results of flow cytometry when mutant iPS cells (KM3-A4) and normal iPS cells (KM3-G1) derived from KM3 were differentiated into hematopoietic progenitor cells. KM3-A4 or KM3-G1 was cultured by EB method to allow for differentiation into hematopoietic progenitor cells.

FIG. 12 shows the results of flow cytometry when MDS-iPSC and Normal iPSC (T-iPS) derived from KM3 and KM5 were differentiated into red blood cells. In the Figure, the horizontal axis shows the ratio of red blood cells (CD235a+).

FIG. 13 shows the results of flow cytometry when abnormal MDS-iPSC (NonT-iPS) and Normal iPSC (T-iPS) derived from KM3 and KM5 were differentiated into neutrophils. In the Figure, the vertical axis shows the ratio of neutrophils (CD66b+) in CD11b+bone marrow cells. 5M-B1 is an iPS cell line showing a normal karyotype.

FIG. 14 shows the karyotype and the results of CGH analysis of MDS-iPS cells. (A) shows the karyotype and the results of CGH analysis of the iPS cell line derived from KM15, and (B) shows the karyotype and the results of CGH analysis of the iPS cell line derived from KM16.

FIG. 15 shows the results of colony formation unit (CFU) assay of hematopoietic progenitor cells (HPC) induced to differentiate from abnormal MDS-iPSC (MDS or MD) and Normal iPSC (Normal or N) derived from KM5, KM15 and KM16. The abbreviations indicated on the right end mean the following: BFU-E: red blood cell, GEMM: mixture, GM: granulocyte, macrophage, G: granulocyte, M: macrophage.

FIG. 16 shows the results of flow cytometry when abnormal MDS-iPSC (MDS or MD) and Normal iPSC (Normal or N) derived from KM3, KM5, KM15 and KM16 were differentiated into red blood cells. In the Figure, the vertical axis shows the ratio of red blood cells (CD235a+). 5M-B1 is an iPS cell line showing a normal karyotype.

FIG. 17 shows the results of flow cytometry when abnormal MDS-iPSC (MDS or MD) and Normal iPSC (Normal or N) derived from KM3, KM5, KM15 and KM16 were differentiated into neutrophils. In the Figure, the vertical axis shows the ratio of neutrophils (CD66b+) in CD11b+bone marrow cells. 5M-B1 is an iPS cell line showing a normal karyotype.

FIG. 18 shows the results of flow cytometry when mutant iPS cell (KM3-A4) and normal iPS cell (KM3-A3) derived from KM3 were differentiated into megakaryocytes.

FIG. 19 shows the observation results of a colony-forming ability-improving effect in MDS-iPSC and Normal iPSC derived from KM5 and KM16 by the application of a p38 inhibitor. The abbreviations indicated on the right end mean the following: BFU-E: red blood cell, GEMM: mixture, GM: granulocyte, macrophage, G: granulocyte, M: macrophage. Application of a p38 inhibitor, SB203580, revealed a colony-forming ability-improving effect.

### DESCRIPTION OF EMBODIMENTS

The “mutant MDS-iPS cells”, “mutant MDS-iPSCs”, “MDS-iPSCs” and the terms analogous thereto, which are used in the present specification, mean iPS cells produced from non-T cells in blood mononuclear cells isolated from a myelodysplastic syndrome patient, which are characterized by low colony-forming ability of hematopoietic progenitor cells induced from iPS cells, and low differentiation potency of the iPS cells into the hematopoietic cells (e.g., red blood cell, neutrophil, megakaryocyte). Preferred are iPS cells having cellular pathology unique to myelodysplastic syndrome, for example, chromosome abnormality. The “mutant MDS-iPS cells”, “mutant MDS-iPSCs” and “MDS-iPSCs”, which are used in the present specification, can also be interpreted to mean “abnormal MDS-iPS cells” in contrast to the following “normal MDS-iPS cells”, “normal MDS-iPSCs” and “Normal iPSCs”.

On the other hand, the “normal MDS-iPS cell”, “normal MDS-iPSCs”, “Normal iPSC” and the terms analogous thereto, which are used in the present specification, mean iPS cells free of cellular pathology unique to myelodysplastic syndrome, from among the iPS cells produced from somatic cells of a myelodysplastic syndrome patient. The iPS cells produced from the T cells in the blood mononuclear cells isolated from a myelodysplastic syndrome patient are free of the cellular pathology unique to myelodysplastic syndrome such as chromosome abnormality and the like, and the hematopoietic progenitor cells induced from the iPS cells have normal colony-forming ability and differentiation potency into hematopoietic cells. Therefore, preferred is an iPS cell produced from T cells.

### Production Method of iPS Cells

In the present invention, an iPS cell is an artificial stem cell derived from a somatic cell, which has nearly the same characteristics as those of ES cells, for example, differentiation pluripotency and the potential for proliferation by self-renewal, and that can be prepared by transferring a certain nuclear reprogramming substance, in the form of a protein or nucleic acid encoding same, to a somatic cell, or by increasing the expression of mRNA and protein of the nuclear reprogramming substance, which is endogenous, by a medicament [K. Takahashi and S. Yamanaka (2006) Cell, 126: 663-676; K. Takahashi et al. (2007) Cell, 131: 861-872; J. Yu et al. (2007) Science, 318: 1917-1920; M. Nakagawa et al. (2008) Nat. Biotechnol., 26: 101-106; WO 2007/069666 and WO 2010/068955). The nuclear reprogramming substance is not particularly limited and may be any gene specifically expressed in ES cells, or a gene that plays a key role in the maintenance of the undifferentiated state of ES cells, or a gene product thereof. Examples thereof include Oct3/4, Klf4, Klf1, Klf2, Klf5, Sox2, Sox1, Sox3, Sox15, Sox17, Sox18, c-Myc, L-Myc, N-Myc, TERT, SV40 Large T antigen, HPV16 E6, HPV16 E7, Bmil, Lin28, Lin28b, Nanog, Esrrb, Esrrg and Glis1. These reprogramming substances may be used in combination when establishing iPS cells. For example, a combination comprising at least one, two or three of the above-mentioned reprogramming substances may be used, with preference given to a combination comprising four.

Information on the nucleotide sequences of the mouse and human cDNAs and amino acid sequences of proteins encoded thereby of the above-described nuclear reprogramming substances is available with reference to the NCBI accession numbers shown in WO 2007/069666. Information on the mouse and human cDNA and amino acid sequences of L-Myc, Lin28, Lin28b, Esrrb, Esrrg and Glis1 is available with reference to the NCBI accession numbers shown below. Those skilled in the art are able to prepare a desired nuclear reprogramming substance by a conventional method on the basis of the information on the cDNA sequence or amino acid sequence thereof.

| Name of gene | mouse | human |
|---|---|---|
| L-Myc | NM_008506 | NM_001033081 |
| Lin28 | NM_145833 | NM_024674 |
| Lin28b | NM_001031772 | NM_001004317 |
| Esrrb | NM_011934 | NM_004452 |
| Esrrg | NM_011935 | NM_001438 |
| Glis1 | NM_147221 | NM_147193 |

These nuclear reprogramming substances may be introduced into a somatic cell in the form of protein, for example, by methods such as lipofection, conjugation with cell-penetrating peptide, microinjection and the like. Alternatively, they can be introduced into a somatic cell in the form of DNA, for example, using vectors such as virus, plasmid and artificial chromosome and by methods such as lipofection, liposome method and microinjection. Examples of viral vectors include retrovirus vectors, lentivirus vectors (both Cell, 126, pp. 663-676, 2006; Cell, 131, pp. 861-872, 2007; Science, 318, pp. 1917-1920, 2007), adenovirus vectors (Science, 322, 945-949, 2008), adeno-associated virus vectors, Sendai virus vectors (Proc. Jpn. Acad. Ser. B. Phys. Biol. Sci. 85, 348-62, 2009) and the like. Examples of the artificial chromosome vector include human artificial chromosome (HAC), yeast artificial chromosome (YAC), bacterial artificial chromosome (BAC, PAC) and the like. As regards plasmid, plasmids for mammalian cells can be used (Science, 322: 949-953, 2008). The vectors may contain regulatory sequences such as a promoter, enhancer, ribosome-binding sequence, terminator or polyadenylation site to allow a nuclear reprogramming substance to be expressed. Examples of the promoter to be used include EF1α promoter, CAG promoter, SRα promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like. Of these, EF1α promoter, CAG promoter, MoMuLV LTR, CMV promoter, SRα promoter and the like can be mentioned. The expression vector can further contain, as required, a selectable marker sequence such as a drug resistance gene (e.g., kanamycin resistance gene, ampicillin resistance gene, puromycin resistance gene and the like), the thymidine kinase gene or diphtheria toxin gene, a reporter gene sequence such as of green fluorescent protein (GFP), β-glucuronidase (GUS) or FLAG, and the like. The above-mentioned vector may have a loxP sequence placed at both ends of the gene that encodes the nuclear reprogramming substance or of a promoter and the gene connected thereto, to enable resection thereof, after being transferred to somatic cells. In another preferable embodiment, a method including integrating transgene into the chromosome by using transposon, reacting transferase with the cell by using a plasmid vector or adenovirus vector, and completely removing the transgene from the chromosome is used. Examples of the preferable transposon include piggyBac which is a lepidopterous insect-derived transposon and the like (Kaji, K. et al., (2009), Nature, 458: 771-775, Woltjen et al., (2009), Nature, 458: 766-770, WO 2010/012077). Furthermore, the vector may also contain the origins and sequences relating to the replication of lymphotrophic herpes virus, BK virus and bovine papillomavirus, so as to be replicated to exist episomally even in the absence of integration into the chromosome. For example, it may contain EBNA-1 and oriP or Large T and SV40ori sequences (WO 2009/115295, WO 2009/157201 and WO 2009/149233). To simultaneously introduce multiple nuclear reprogramming substances, an expression vector causing polycistronic expression may also be used. For polycistronic expression, sequences encoding the gene may be linked by IRES or a foot-and-mouth disease virus (FMDV) 2A coding region (Science, 322:949-953, 2008 and WO 2009/092042, WO 2009/152529).

To increase iPS cell induction efficiency in nuclear reprogramming, in addition to the above-described factors, for example, histone deacetylase (HDAC) inhibitors [e.g., low-molecular inhibitors such as valproic acid (VPA) (Nat. Biotechnol., 26(7): 795-797 (2008)), trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29mer shRNA constructs against HDAC1 (OriGene) and the like), and the like], DNA methyltransferase inhibitors (e.g., 5′-azacytidine) [Nat. Biotechnol., 26(7): 795-797 (2008)], G9a histone methyltransferase inhibitors [e.g., low-molecular inhibitors such as BIX-01294 (Cell Stem Cell, 2: 525-528 (2008)), nucleic acid-based expression inhibitors such as siRNAs and shRNAs against G9a (e.g., G9a siRNA (human) (Santa Cruz Biotechnology) and the like) and the like], L-channel calcium agonists (e.g., Bayk8644) [Cell Stem Cell, 3, 568-574 (2008)], p53 inhibitors [e.g., siRNA and shRNA against p53 (Cell Stem Cell, 3, 475-479 (2008)), Wnt Signaling activator (e.g., soluble Wnt3a) [Cell Stem Cell, 3, 132-135 (2008)], growth factors such as LIF, bFGF etc., ALK5 inhibitors (e.g., SB431542) [Nat Methods, 6: 805-8 (2009)], a mitogen-activated protein kinase signaling inhibitor, a glycogen synthase kinase-3 inhibitor [PloS Biology, 6(10), 2237-2247 (2008)], miRNAs such as miR-291-3p, miR-294, and miR-295 [R. L. Judson et al., Nat. Biotechnol., 27:459-461 (2009)], and the like can be used.

As a medicament to be used for a method of increasing expression of an endogenous protein of a nuclear reprogramming substance by using a medicament, 6-bromoindirubin-3′-oxime, indirubin-5-nitro-3′-oxime, valproic acid, 2-(3-(6-methylpyridin-2-yl)-1H-pyrazol-4-yl)-1,5-naphthyridine, 1-(4-methylphenyl)-2-(4,5,6,7-tetrahydro-2-imino-3(2H)-benzothiazolyl)ethanone HBr(pifithrin-alpha), prostaglandin J2 and prostaglandin E2 and the like can be recited (WO 2010/068955).

Examples of culture media for iPS cell induction include (1) a DMEM, DMEM/F12 or DME medium containing 10 to 15% FBS (these media can further contain LIF, penicillin/streptomycin, puromycin, L-glutamine, non-essential amino acids, β-mercaptoethanol and the like as appropriate), (2) an ES cell culture medium containing bFGF or SCF, for example, a mouse ES cell culture medium (e.g., TX-WES medium, Thromb-X) or a primate ES cell culture medium [e.g., primate (human and monkey) ES cell culture medium (ReproCELL, Kyoto, Japan), mTeSR-1], and the like.

As an example of the culture method, somatic cells and a nuclear reprogramming substance (DNA or protein) are brought into contact with each other in a DMEM or DMEM/F12 medium containing 10% FBS and cultured at 37° C. in the presence of 5% CO₂ for about 4 to 7 days, after which the cells are re-seeded onto feeder cells (e.g., STO cells, SNL cells and the like, treated with mitomycin C), and again cultured using a bFGF-containing primate ES cell culture medium, starting about 10 days after contact of the somatic cells and the nuclear reprogramming substance, whereby ES cell-like colonies can be produced in about 30 to about 45 days or more after the contact. To increase the efficiency of iPS cell induction, the cells may be cultured under conditions involving a low oxygen concentration of 5-10%.

As an alternative culture method thereof, the cells may be cultured on feeder cells (e.g., STO cells, SNL cells and the like, treated with mitomycin C), using a DMEM medium containing 10% FBS (this can further contain LIF, penicillin/streptomycin, puromycin, L-glutamine, non-essential amino acids, β-mercaptoethanol and the like as appropriate), whereby ES-like colonies can be produced after about 25 to about 30 days or more.

During the period of culture, the medium is replaced with a fresh supply of the same medium once daily starting on day 2 of culture. Although the number of somatic cells used for nuclear reprogramming is not subject to limitations, it falls in the range of about 5×10³ to about 5×10⁶ cells per 100 cm² of culture dish.

When a gene containing a drug resistance gene is used as a marker gene, cells that express the marker gene can be selected by culture using a medium containing the corresponding drug (selection medium). Cells that express the marker gene can be detected by making an observation using a fluorescence microscope for a fluorescent protein gene as the marker gene, by adding a luminogenic substrate for a luminescence enzyme gene as the marker gene, and by adding a color developing substrate for a color developing enzyme gene as the marker gene.

Any cells other than the germ cells obtained from mammals (e.g., human, mouse, monkey, swine, rat etc.) can be used as the “somatic cells” used in the present invention. Examples include keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), constrictive cells (e.g., smooth muscle cells), cells of the blood and the immune system (e.g., T lymphocytes), sense-related cells (e.g., rod cells), autonomic neurons (e.g., cholinergic neurons), sustentacular cells of sensory organs and peripheral neurons (e.g., satellite cells), neurons and glia cells in the central nervous system (e.g., astroglia cells), pigment cells (e.g., retinal pigment epithelial cells), progenitor cells (tissue progenitor cells) thereof and the like. There is no limitation on the degree of cell differentiation, the age of the animal from which cells are collected and the like; even undifferentiated progenitor cells (including somatic stem cells) and terminally differentiated mature cells can be used alike as sources of somatic cells in the present invention. Here, examples of undifferentiated progenitor cells include tissue stem cells (somatic stem cells) such as neural stem cells, hematopoietic stem cells, mesenchymal stem cells, and dental pulp stem cells. In the present invention, non-T cells in blood mononuclear cells are desirably used to produce iPS cells having cellular pathology unique to myelodysplastic syndrome. As used herein, the non-T cells mean cells other than T cell among the mononuclear cells in the blood, and are characterized in that, for example, all markers of CD3, CD4, CD8 and CD9 are negative. Non-T cells may be a cell population which is concentrated by culturing mononuclear cells in the blood in a culture medium containing IL-3, IL-6, G-CSF and GM-CSF. In the present invention, T cells in the blood are desirably used as somatic cells, to produce iPS cells, which are free of cellular pathology unique to myelodysplastic syndrome, from a myelodysplastic syndrome patient. As used herein, T cells are characterized in that, for example, at least one marker selected from CD3, CD4, CD8 and CD9 is positive. T cells may also be a cell population which is concentrated by culturing mononuclear cells in the blood in a culture medium containing IL-2, CD3 antibody and CD28 antibody.

In the present invention, while a mammalian individual from which the somatic cells are to be collected is not particularly limited, it is preferably human.

When hematopoietic progenitor cells induced to differentiate from iPS cells are used for a method of screening for a therapeutic and/or prophylactic drug for myelodysplastic syndrome, the iPS cells are desirably prepared by collecting somatic cells from a patient known to have been affected with myelodysplastic syndrome. In this case, the iPS cells desirably have chromosome abnormality unique to myelodysplastic syndrome. The chromosome abnormality of myelodysplastic syndrome includes partial abnormality of chromosome and numerical abnormality of chromosome. Examples of the partial abnormality of chromosome include, but are not limited to, duplication, deletion, translocation and the like. Examples of the numerical abnormality of chromosome include “monosomy” or presence of one chromosome, “trisomy” or presence of three chromosomes, “tetrasomy” or presence of four chromosomes, and “pentasomy” or presence of five chromosomes. Examples of the chromosome abnormality of myelodysplastic syndrome include, but are not limited to, lack of a part or whole part of long arm of chromosome 4 (4q) (i.e., decrease in copy number), lack of a part or whole part of long arm of chromosome 5 (5q) (i.e., decrease in copy number), lack of a part or whole part of long arm of chromosome 7 (7q) (i.e., decrease in copy number), duplication of a part or whole part of short arm of chromosome 9 (9p) (i.e., increase in copy number), lack of a part or whole part of long arm of chromosome 11 (11q) (i.e., decrease in copy number), lack of a part or whole part of short arm of chromosome 17 (17p) (i.e., increase or decrease in copy number), lack of a part or whole part of short arm of chromosome 18 (18p) (i.e., decrease in copy number), lack of a part or whole part of long arm of chromosome 20 (20q) (i.e., decrease in copy number), translocation between long arm of chromosome 3 and long arm of chromosome 21 and the like.

In the present invention, iPS cells to be used for a method of screening for a therapeutic and/or prophylactic drug for myelodysplastic syndrome are, for example, iPS cells produced from non-T cells collected from a patient known to have been affected with myelodysplastic syndrome. Interestingly, hematopoietic progenitor cells induced to differentiate from iPS cells produced from non-T cells collected from a myelodysplastic syndrome patient and having none of the aforementioned chromosome abnormalities may express pathology of myelodysplastic syndrome.

Hematopoietic progenitor cells induced to differentiate from iPS cells as a control necessary for this screening method may be induced from normal somatic cells collected from a patient known to have been affected with myelodysplastic syndrome, or induced from somatic cells derived from normal individual. To arrange other conditions, iPS cells prepared from normal somatic cells collected from a myelodysplastic syndrome patient used for the production of iPS cells having the chromosome abnormality unique to myelodysplastic syndrome are desirably used as control iPS cells. More preferably, iPS cells produced from T cells of a myelodysplastic syndrome patient are used as a control for the production of hematopoietic progenitor cells.

### Method of Differentiation Induction into Hematopoietic Progenitor Cell

Examples of the method for inducing differentiation of pluripotent stem cells into hematopoietic progenitor cells include, but are not limited to, a method including embryoid body formation and cytokine addition (Chadwick et al. Blood 2003, 102:906-15, Vijayaragavan et al. Cell Stem Cell 2009, 4:248-62 and Saeki et al. Stem Cells 2009, 27:59-67), a method including coculture with xenogeneic stromal cells (Niwa A et al. J Cell Physiol. 2009 November; 221(2):367-77.), a method including use of a serum-free medium (WO 2011/115308) and the like.

In the present invention, the “hematopoietic progenitor cell” means a cell that shows more progress of differentiation than hematopoietic stem cells, and has a determined direction of cell differentiation. While these cells can be detected by the expression of a marker such as KDR, CD34, CD90 and CD117, the marker is not limited thereto. Preferred is a CD43 positive, CD34 positive, and CD38 negative cell. On the other hand, the “hematopoietic stem cell” means a cell that has an ability to produce mature blood cells such as T cell, B cell, red blood cell, platelet, eosinophil, monocyte, neutrophil, basophil and the like, and also has self-replication competence. The “hematopoietic progenitor cell” in the present specification includes “hematopoietic stem cell” unless particularly indicated.

The hematopoietic progenitor cells induced to differentiate in the present invention may be provided as a cell population containing other cell type or as a purified population.

In the induction of hematopoietic progenitor cells in the present invention, pluripotent stem cells such as ES cell, iPS cell and the like may be separated by any method, and induced by floating culture, or may be induced by adhesion culture using a coating-treated culture dish. Examples of the separation method of human pluripotent stem cells include a physical separation method, and a separation method using a separation solution (e.g., Accutase (™) and Accumax (™) and the like) having protease activity and collagenase activity, or a separation solution having collagenase activity alone. Preferably, a method including dissociating human pluripotent stem cells by using a separation solution having protease activity and collagenase activity (particularly preferably, Accutase (™)) to physically disperse them as fine single cells is used. The human pluripotent stem cells used here are preferably colonies cultured up to 80% confluence to the dish used. As a separation method of the mouse pluripotent stem cells, a separation method using 0.25% trypsin/EDTA can be mentioned.

As used herein, floating culture refers to forming embryoid bodies by culturing cells in a non-adhesion state to a culture dish, and is not particularly limited. A culture dish free of an artificial treatment to improve adhesiveness to the cells (e.g., coating treatment with extracellular matrix and the like), or a culture dish treated to artificially suppress adhesion (e.g., coating treatment with polyhydroxyethylmethacrylic acid (poly HEMA)) is used to perform the culture.

When adhesion culture is used in the present invention, cells may be cultured on feeder cells or in any medium in a coating-treated culture dish. The feeder cell here means other cell that plays an auxiliary role of adjusting the culture conditions for the object cell. For example, cells obtained from the AGM region of mammal fetus (e.g., AGM-S3 cell line: JP-A-2001-37471), mouse mesenchymal cell (e.g., C3H10T1/2 cell line: available from Riken BioResource Center), or interstitial cell (stromal cell) derived from the bone marrow (e.g., OP9 cell line) can be used. Examples of the coating agent include Matrigel (BD), collagen, gelatin, laminin, heparan sulfate proteoglycan, entactin, and a combination of these.

In the present invention, a medium for inducing hematopoietic progenitor cells can be prepared using a medium used for culturing animal cells as a basal medium. Examples of the basal medium include IMDM medium, Medium 199 medium, Eagle's Minimum Essential Medium (EMEM), αMEM medium, Dulbecco's modified Eagle's Medium (DMEM), Ham's F12 medium, RPMI 1640 medium, Fischer's medium, StemPro34 (invitrogen), a mixed medium of these and the like. The medium may contain a serum, or may be serum-free. Where necessary, the medium may contain, for example, one or more serum replacements such as albumin, transferrin, Knockout Serum Replacement (KSR) (serum replacement of FBS during culture of ES cells), N2 supplement (Invitrogen), B27 supplement (Invitrogen), fatty acid, insulin, collagen precursor, trace element, 2-mercaptoethanol (2ME), thiolglycerol and the like, and can also contain one or more substances from lipid, amino acid, L-glutamine, Glutamax (Invitrogen), non-essential amino acid, vitamin, growth factor, low-molecular compound, antibiotic, antioxidant, pyruvic acid, buffering agent, inorganic salts and the like.

When adhesion culture is used in the present invention, a medium for differentiation into hematopoietic progenitor cells preferably contains a vascular endothelial growth factor (VEGF). The concentration of VEGF in the medium is not limited and is, for example, 1 ng/ml, 5 ng/ml, 10 ng/ml, 20 ng/ml, 30 ng/ml, 40 ng/ml, 50 ng/ml, 75 ng/ml, 100 ng/ml, 200 ng/ml, 300 ng/ml, 400 ng/ml, 500 ng/ml, 1 pg/ml, preferably, 20 ng/ml.

When adhesion culture is used in the present invention, a more preferable medium can be αMEM medium containing 10% FBS, VEGF, transferrin, L-glutamine, α-monothioglycerol (MTG) and ascorbic acid (also referred to as HPC differentiation medium).

When adhesion culture is used in the present invention, the number of culture days is, for example, not less than 20 days, preferably, not less than 12 days and not more than 14 days, particularly preferably, 13 days, of culture.

When adhesion culture is used in the present invention, a step of removing feeder cells to concentrate and purify hematopoietic progenitor cells can be included. This step can be achieved by detaching hematopoietic progenitor cells together with feeder cells from a culture dish and recovering and removing only the feeder cells. Examples of the method for detaching from a culture dish include, but are not limited to, a physical separation method, and a separation method using a separation solution having protease activity and collagenase activity, or a separation solution having collagenase activity alone. For example, a method using collagenase Type IV and/or Trypsin/EDTA is used. As a separation method of hematopoietic progenitor cells, 0.05% trypsin/EDTA is preferably used.

When floating culture is used in the present invention, differentiation into hematopoietic progenitor cells may be induced by, for example, the following steps.
(i) a step of forming EB
(ii) a step of forming primitive streak/mesoderm
(iii) a step of specification into hematopoietic progenitor cells

To induce object cells in each of the above-mentioned steps, or to achieve the object in each step, a medium obtained by adding any necessary substances to the basic medium can be used. For example, a medium containing the following substances is used in each step.
(i) BMP-4
(ii) bFGF
(iii) VEGF, bFGF, IL-6, IL-3, IL-11, SCF and FLT3L

The basal medium used in the aforementioned steps (i) to (iii) is preferably StemPro-34 supplemented with L-glutamic acid, thioglycerol and ascorbic acid.

The concentration of BMP-4 in the medium of the aforementioned step (i) may be any as long as EB can be formed, it is, for example, 1 ng/ml, 5 ng/ml, 10 ng/ml, 20 ng/ml, 30 ng/ml, 40 ng/ml, 50 ng/ml, 75 ng/ml, 100 ng/ml, preferably, 10 ng/ml.

The concentration of bFGF in the medium of the aforementioned step (ii) is not limited and is, for example, 100 pg/ml, 250 pg/ml, 500 pg/ml, 750 pg/ml, 1 ng/ml, 5 ng/ml, 10 ng/ml, 20 ng/ml, preferably, 1 ng/ml.

The concentration of VEGF in the medium of the aforementioned step (iii) is not limited and is, for example, 1 ng/ml, 5 ng/ml, 10 ng/ml, 20 ng/ml, 30 ng/ml, 40 ng/ml, 50 ng/ml, 75 ng/ml, 100 ng/ml, preferably, 10 ng/ml.

The concentration of IL-6 in the medium of the aforementioned step (iii) is not limited and is, for example, 1 ng/ml, 5 ng/ml, 10 ng/ml, 20 ng/ml, 30 ng/ml, 40 ng/ml, 50 ng/ml, 75 ng/ml, 100 ng/ml, preferably, 10 ng/ml.

The concentration of IL-3 in the medium of the aforementioned step (iii) is not limited and is, for example, 1 ng/ml, 5 ng/ml, 10 ng/ml, 20 ng/ml, 30 ng/ml, 40 ng/ml, 50 ng/ml, 75 ng/ml, 100 ng/ml, preferably, 40 ng/ml.

The concentration of IL-11 in the medium of the aforementioned step (iii) is not limited and is, for example, 500 pg/ml, 750 pg/ml, 1 ng/ml, 5 ng/ml, 10 ng/ml, 20 ng/ml, 30 ng/ml, 40 ng/ml, 50 ng/ml, preferably, 5 ng/ml.

The concentration of SCF in the medium of the aforementioned step (iii) is not limited and is, for example, 1 ng/ml, 25 ng/ml, 50 ng/ml, 75 ng/ml, 100 ng/ml, 200 ng/ml, 300 ng/ml, 400 ng/ml, 500 ng/ml, preferably, 100 ng/ml.

The concentration of FLT3L in the medium of the aforementioned step (iii) is not limited and is, for example, 1 ng/ml, 25 ng/ml, 50 ng/ml, 75 ng/ml, 100 ng/ml, 200 ng/ml, 300 ng/ml, 400 ng/ml, 500 ng/ml, preferably, 100 ng/ml.

The culture period of the aforementioned step (i) is, for example, not more than 5 days, preferably, 1-3 days, particularly preferably 1 day, of culture.

The culture period of the aforementioned step (ii) is, for example, not more than 10 days, preferably, 1-5 days, particularly preferably 3 days, of culture.

The culture period of the aforementioned step (iii) is, for example, not more than 10 days, preferably, 2-6 days, particularly preferably 4 days, of culture.

After the completion of the above-mentioned production step (ii), a step for refining and purifying the cell population of mesoderm can be performed. For refinement and purification of the cell population of mesoderm, any method can be used as long as it can separate the cell population of mesoderm from a population of cells including the cell population of mesoderm at high purity and, for example, refinement and purification by FACS can be mentioned. In the present invention, to prevent the presence of undifferentiated cells in the refined and purified population of mesoderm, the cells can be further selected using SSEA-1 negativity (i.e., SSEA-1−) as an index. A step for selecting Flk1 positive (i.e., Flk1+) and SSEA-1− cells can be performed simultaneously or performed as a separate step. For example, Flk1+/SSEA-1− cells can be simultaneously selected using FACS.

In this step, the culture temperature is not limited to the below, but is about 30-40° C., preferably about 37° C., and culture is performed under an atmosphere of CO₂-containing air. The concentration of CO₂ is about 2-5%, preferably 5%.

In this step, the medium may further contain a ROCK inhibitor. Particularly when this step includes a step of dispersing human pluripotent stem cells into single cells, the medium preferably contains a ROCK inhibitor. The ROCK inhibitor is not particularly limited as long as it can suppress the function of Rho kinase (ROCK) and, for example, Y 27632 can be used in the present invention.

### Method of Differentiation Induction into Red Blood Cell

Examples of the method for inducing differentiation of pluripotent stem cells into hematopoietic cells, particularly red blood cells include, but are not limited to, a method including embryoid body formation and cytokine addition (Chadwick et al. Blood 2003, 102:906-15, Vijayaragavan et al. Cell Stem Cell 2009, 4:248-62 and Saeki et al. Stem Cells 2009, 27:59-67), a method including coculture with xenogeneic stromal cells (Niwa A et al. J Cell Physiol. 2009 November; 221(2):367-77.), a method including use of a serum-free medium (WO 2011/115308) and the like.

In the present invention, the “red blood cell” means a cell rich in hemoglobin. While the “red blood cell” can be detected based on the expression of a marker of hemoglobin such as α-globin, ε-globin, γ-globin, β-globin and CD235a in the present invention, the marker is not limited to these. A preferable marker for matured red blood cell can be α-globin or β-globin. When red blood cell is separated by FACS, a preferable marker can be CD235a.

The red blood cells induced to differentiate in the present invention may be provided as a cell population containing other cell type or as a purified population.

In this step, the aforementioned hematopoietic progenitor cells may be separated by any method, and cultured by floating culture, or may be adhesion cultured using a coating-treated culture dish. As a culture method in the present invention, floating culture is preferably employed. In this step, hematopoietic progenitor cells may be separated and used, or the state obtained by the aforementioned method may be used as it is.

The floating culture refers to culture of cells in a non-adhesion state to a culture dish, and is not particularly limited. A culture dish free of an artificial treatment to improve adhesiveness to the cells (e.g., coating treatment with extracellular matrix and the like), or a culture dish treated to artificially suppress adhesion (e.g., coating treatment with polyhydroxyethylmethacrylic acid (poly HEMA)) is used to perform the culture.

In adhesion culture, cells are cultured on feeder cells or in any medium in a coating-treated culture dish. The feeder cell means other cell that plays an auxiliary role of adjusting the culture conditions for the object cell. Examples of the feeder cell in this step include interstitial cell (stromal cell) derived from the bone marrow, and OP9 cell can be used preferably. Examples of the coating agent include Matrigel (BD), collagen, gelatin, laminin, heparan sulfate proteoglycan, entactin, and a combination of these.

The medium in this step can be prepared using a medium used for culturing animal cells as a basal medium. Examples of the basal medium include IMDM medium, Medium 199 medium, Eagle's Minimum Essential Medium (EMEM), αMEM medium, Dulbecco's modified Eagle's Medium (DMEM), Ham's F12 medium, RPMI 1640 medium, Fischer's medium, StemPro34 medium, a mixed medium of these and the like. Preferred is StemPro-34 medium. The medium may contain a serum, or may be serum-free. Where necessary, the medium may contain, for example, one or more serum replacements such as albumin, transferrin, Knockout Serum Replacement (KSR) (serum replacement of FBS during culture of ES cells), N2 supplement (Invitrogen), B27 supplement (Invitrogen), EasyDiff™ Erythroid Supplement (Lonza), fatty acid, insulin, collagen precursor, trace element, 2-mercaptoethanol (2ME), thiolglycerol and the like, and can also contain one or more substances from lipid, amino acid, L-glutamine, Glutamax (Invitrogen), non-essential amino acid, vitamin, growth factor, low-molecular compound, antibiotic, antioxidant, pyruvic acid, buffering agent, inorganic salts and the like.

The medium in this step may further contain a ROCK inhibitor. Particularly when this step includes a step of dispersing human pluripotent stem cells into single cells, the medium preferably contains a ROCK inhibitor. The ROCK inhibitor is not particularly limited as long as it can suppress the function of Rho kinase (ROCK) and, for example, Y 27632 can be used in the present invention.

The culture temperature is not limited to the below, but is about 30-40° C., preferably about 37° C., and culture is performed under an atmosphere of CO₂-containing air. The concentration of CO₂ is about 2-5%, preferably 5%.

Examples of the differentiation inducer into red blood cell include stem cell factor (SCF), colony-stimulating factor (CSF), granulocyte colony stimulating factor (Granulocyte-(G-) CSF), erythropoietin (EPO), interleukins, thrombopoietin (TPO) and Flt3 ligand and the like. As used herein, interleukin is a protein secreted by leukocyte, and there are more than 30 kinds including IL-1, IL-2, IL-3, IL-4, IL-5, IL-6, IL-7, IL-8, and IL-9 and the like.

In the present invention, induction of differentiation from hematopoietic progenitor cells into red blood cells may be performed, for example, by the following steps:
(i) a step of culturing hematopoietic progenitor cells in a medium containing VEGF, IL-6, IL-3, IL-11, SCF, FLT3L, EPO and TPO,
(ii) a step of culturing the cells obtained in step (i) in a medium containing IL-3, SCF and EPO, and
(iii) a step of culturing the cells obtained in step (ii) in a medium containing SCF and EPO.

The concentration of a differentiation inducer into red blood cell in the medium may be any as long as the object cell can be induced.

The concentration of VEGF in the medium is not limited and is, for example, 1 ng/ml, 5 ng/ml, 10 ng/ml, 20 ng/ml, 30 ng/ml, 40 ng/ml, 50 ng/ml, 75 ng/ml, 100 ng/ml, preferably, 10 ng/ml.

The concentration of IL-6 in the medium is not limited and is, for example, 1 ng/ml, 5 ng/ml, 10 ng/ml, 20 ng/ml, 30 ng/ml, 40 ng/ml, 50 ng/ml, 75 ng/ml, 100 ng/ml, preferably, 10 ng/ml.

The concentration of IL-3 in the medium is not limited and is, for example, 1 ng/ml, 5 ng/ml, 10 ng/ml, 20 ng/ml, 30 ng/ml, 40 ng/ml, 50 ng/ml, 75 ng/ml, 100 ng/ml, preferably, 40 ng/ml.

The concentration of IL-11 in the medium is not limited and is, for example, 500 pg/ml, 750 pg/ml, 1 ng/ml, 5 ng/ml, 10 ng/ml, 20 ng/ml, 30 ng/ml, 40 ng/ml, 50 ng/ml, preferably, 5 ng/ml.

The concentration of SCF in the medium is not limited and is, for example, 1 ng/ml, 25 ng/ml, 50 ng/ml, 75 ng/ml, 100 ng/ml, 200 ng/ml, 300 ng/ml, 400 ng/ml, 500 ng/ml, preferably, 100 ng/ml.

The concentration of FLT3L in the medium is not limited and is, for example, 1 ng/ml, 25 ng/ml, 50 ng/ml, 75 ng/ml, 100 ng/ml, 200 ng/ml, 300 ng/ml, 400 ng/ml, 500 ng/ml, preferably, 100 ng/ml.

The concentration of EPO in the medium is not limited and is, for example, 1 U/ml, 2 U/ml, 3 U/ml, 4 U/ml, 5 U/ml, 6 U/ml, 7 U/ml, 8 U/ml, 9 U/ml, 10 U/ml, preferably, 4 U/ml.

The concentration of TPO in the medium is not limited and is, for example, 1 ng/ml, 5 ng/ml, 10 ng/ml, 20 ng/ml, 30 ng/ml, 40 ng/ml, 50 ng/ml, 75 ng/ml, 100 ng/ml, preferably, 50 ng/ml.

The culture period of the aforementioned step (i) is, for example, not more than 10 days, preferably, 2-6 days, particularly preferably 4 days, of culture.

The culture period of the aforementioned step (ii) is, for example, not more than 20 days, preferably, 5-15 days, particularly preferably 10 days, of culture.

The culture period of the aforementioned step (iii) is, for example, not more than 20 days, preferably, 12-16 days, particularly preferably 14 days, of culture.

In the aforementioned step (iii), the medium can also be exchanged during culture with a medium containing SCF and EPO. The culture period in this case is, for example, not more than 12 days, preferably 7-9 days, particularly preferably 8 days, of culture before the medium exchange, and is, for example, not more than 8 days, preferably 5-7 days, particularly preferably 6 days, of culture after the medium exchange.

### Method of Differentiation Induction into Neutrophil

Examples of the method for inducing differentiation of pluripotent stem cells into neutrophils include, but are not limited to, Saeki et al., Stem Cells 27: 59-67, Morishima et al., J Cell Physiol. 2011 May; 226(5):1283-91 and the like.

In the present invention, “neutrophil” means a cell which is one kind of leukocyte, and responsible for immune reactions in bacterial infection and the like. In the present invention, while “neutrophil” can be detected by the expression of a marker such as Ly6g, CD11b and CD66b, the marker is not limited thereto. In neutrophil, a preferable marker can be CD66b.

The neutrophil obtained by differentiation induction in the present invention may be provided as a cell population containing other cell type, or may be a refined population.

In this step, the aforementioned hematopoietic progenitor cells may be cultured by floating culture, or may be adhesion cultured using a coating-treated culture dish. As a culture method in the present invention, adhesion culture is preferably employed.

In floating culture, cells are cultured in a non-adhesion state to a culture dish, and the culture is not particularly limited. A culture dish free of an artificial treatment to improve adhesiveness to the cells (e.g., coating treatment with extracellular matrix and the like), or a culture dish treated to artificially suppress adhesion (e.g., coating treatment with polyhydroxyethylmethacrylic acid (poly HEMA)) is used to perform the culture.

In adhesion culture, cells are cultured on feeder cells or in any medium in a coating-treated culture dish. The feeder cell means other cell that plays an auxiliary role of adjusting the culture conditions for the object cell. Examples of the feeder cell in this step include interstitial cell (stromal cell) derived from the bone marrow and the like, and OP9 cell can be used preferably. Examples of the coating agent include Matrigel (BD), collagen, gelatin, laminin, heparan sulfate proteoglycan, entactin, and a combination of these.

The medium in this step can be prepared using a medium used for culturing animal cells as a basal medium. Examples of the basal medium include IMDM medium, Medium 199 medium, Eagle's Minimum Essential Medium (EMEM), αMEM medium, Dulbecco's modified Eagle's Medium (DMEM), Ham's F12 medium, RPMI 1640 medium, Fischer's medium, a mixed medium of these and the like. Preferred is αMEM medium or IMDM medium. The medium may contain a serum, or may be serum-free. Where necessary, the medium may contain, for example, one or more serum replacements such as albumin, transferrin, Knockout Serum Replacement (KSR) (serum replacement of FBS during culture of ES cells), N2 supplement (Invitrogen), B27 supplement (Invitrogen), EasyDiff™ Myeloid Supplement (Lonza), fatty acid, insulin, collagen precursor, trace element, 2-mercaptoethanol (2ME), 3′-thiolglycerol and the like, and can also contain one or more substances from lipid, amino acid, L-glutamine, Glutamax (Invitrogen), non-essential amino acid, vitamin, growth factor, low-molecular compound, antibiotic, antioxidant, pyruvic acid, buffering agent, inorganic salts and the like.

A medium for differentiation into neutrophils preferably contains GM-CSF and/or G-CSF. The concentration of GM-CSF in the medium is not limited and is, for example, 1 ng/ml, 10 ng/ml, 50 ng/ml, 100 ng/ml, 150 ng/ml, 175 ng/ml, 200 ng/ml, 225 ng/ml, 250 ng/ml, 300 ng/ml, 400 ng/ml, 500 ng/ml, 1 μg/ml, preferably, 200 ng/ml. The concentration of G-CSF in the medium is not limited and is, for example, 1 ng/ml, 10 ng/ml, 25 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 175 ng/ml, 200 ng/ml, 300 ng/ml, 400 ng/ml, 500 ng/ml, 1 μg/ml, preferably, 100 ng/ml.

The culture temperature is not limited to the below, but is about 30-40° C., preferably about 37° C., and culture is performed under an atmosphere of CO₂-containing air. The concentration of CO₂ is about 2-5%, preferably 5%. The number of culture days is, for example, not more than 30 days, preferably, 18-25 days, particularly preferably 23 days, of culture.

In the present invention, induction of differentiation from hematopoietic progenitor cells into neutrophils may be performed, for example, by the following steps:
(i) a step of inducing bone marrow progenitor cells from hematopoietic progenitor cells
(ii) a step of inducing neutrophils from the bone marrow progenitor cells

To induce object cells in each of the above-mentioned steps, or to achieve the object in each step, a medium added with any necessary substances can be used. For example, a medium containing the following substances is used in each step.
(i) GM-CSF
(ii) G-CSF

A preferable medium in step (i) can be, for example, αMEM medium containing 10% FBS, 5.5 mg/ml human transferrin, 2 mM L-glutamine, 0.5 mM α-monothioglycerol, 50 μg/ml ascorbic acid, and 200 ng/ml GM-CSF (also referred to as medium for proliferation of pluripotent bone marrow progenitor cell).

A preferable medium in step (ii) can be IMDM medium containing, for example, 20% FBS and 100 ng/ml G-CSF (also referred to as neutrophil differentiation medium).

The culture period of (i) is, for example, not more than 5 days, preferably, 1-3 days, particularly preferably 2 days, of culture. The culture period of (ii) is, for example, not more than 15 days, preferably, 5-12 days, particularly preferably 9 or 10 days, of culture.

### Method of Differentiation Induction into Megakaryocyte

Examples of the method for inducing differentiation of pluripotent stem cells into megakaryocytes include, but are not limited to, WO 2008/041370, WO 2009/122747, Takayama et al., Blood, 111: 5298-5306 2008 and the like.

In the present invention, “megakaryocyte” is one kind of hematopoietic cell, and means a cell having platelet productivity. The “megakaryocyte” in the present invention may be a multinucleated cell and includes, for example, a cell characterized by being CD41a positive, CD42a positive and CD42b positive. The multinucleated megakaryocyte refers to a cell or cell population having a relatively increased nuclear number as compared to hematopoietic progenitor cells. For example, when the nucleus of a hematopoietic progenitor cell to be the basis of megakaryocyte is 2N, a cell of 4N or above is referred to as a multinucleated cell.

The megakaryocytes induced to differentiate in the lo present invention may be provided as a cell population containing other cell type or as a purified population.

In this step, the aforementioned hematopoietic progenitor cells may be cultured by floating culture, or may be adhesion cultured using a coating-treated culture dish. As a culture method in the present invention, adhesion culture is preferably employed.

In floating culture, cells are cultured in a non-adhesion state to a culture dish, and the culture is not particularly limited. A culture dish free of an artificial treatment to improve adhesiveness to the cells (e.g., coating treatment with extracellular matrix and the like), or a culture dish treated to artificially suppress adhesion (e.g., coating treatment with polyhydroxyethylmethacrylic acid (poly HEMA)) is used to perform the culture.

In adhesion culture, cells are cultured on feeder cells or in any medium in a coating-treated culture dish. The feeder cell means other cell that plays an auxiliary role of adjusting the culture conditions for the object cell. The feeder cell in this step is not particularly limited as long as megakaryocyte can be induced and examples thereof include C3H10T1/2 (Katagiri T, et al., Biochem Biophys Res Commun. 172, 295-299 (1990)), interstitial cell (stromal cell) derived from the bone marrow and the like, and OP9 cell can be used preferably. Examples of the coating agent include Matrigel (BD), collagen, gelatin, laminin, heparan sulfate proteoglycan, entactin, and a combination of these.

The medium in this step can be prepared using a medium used for culturing animal cells as a basal medium. Examples of the basal medium include IMDM medium, Medium 199 medium, Eagle's Minimum Essential Medium (EMEM), α MEM medium, Dulbecco's modified Eagle's Medium (DMEM), Ham's F12 medium, RPMI 1640 medium, Fischer's medium, a mixed medium of these and the like. Preferred is αMEM medium. The medium may contain a serum, or may be serum-free. Where necessary, the medium may contain, for example, one or more serum replacements such as albumin, transferrin, Knockout Serum Replacement (KSR) (serum replacement of FBS during culture of ES cells), N2 supplement (Invitrogen), B27 supplement (Invitrogen), EasyDiff™ Megakaryocyte Supplement (Lonza), fatty acid, insulin, collagen precursor, trace element, 2-mercaptoethanol (2ME), 3′-thiolglycerol and the like, and can also contain one or more substances from lipid, amino acid, L-glutamine, Glutamax (Invitrogen), non-essential amino acid, vitamin, growth factor, low-molecular compound, antibiotic, antioxidant, pyruvic acid, buffering agent, inorganic salts and the like.

A medium for differentiation into megakaryocytes preferably contains TPO and/or SCF. The concentration of TPO in the medium is not limited and is, for example, 1 ng/ml, 10 ng/ml, 25 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 175 ng/ml, 200 ng/ml, 300 ng/ml, 400 ng/ml, 500 ng/ml, 1 μg/ml, preferably, 100 ng/ml. The concentration of SCF in the medium is not limited and is, for example, 1 ng/ml, 10 ng/ml, 25 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 175 ng/ml, 200 ng/ml, 300 ng/ml, 400 ng/ml, 500 ng/ml, 1 μg/ml, preferably, 100 ng/ml.

A preferable medium in the present invention can be, for example, αMEM medium containing 10% FBS, 5.5 mg/ml human transferrin, 2 mM L-glutamine, 0.5 mM α-monothioglycerol, 50 μg/ml ascorbic acid and 200 ng/ml GM-CSF (also referred to as megakaryocyte differentiation medium).

The culture temperature is not limited to the below, but is about 30-40° C., preferably about 37° C., and culture is performed under an atmosphere of CO₂-containing air. The concentration of CO₂ is about 2-5%, preferably 5%. The number of culture days is, for example, not more than 20 days, preferably, 5-15 days, particularly preferably 10 days, of culture. It is also possible to add a medium as appropriate during the culture period or exchange a part or whole part of the medium.

### Screening Method of Therapeutic and/or Prophylactic Drugs for Acute Myeloid Leukemia or Myelodysplastic Syndrome (MDS)

The present invention provides a method comprising contacting hematopoietic progenitor cells derived from iPS cells obtained as mentioned above with a test substance, and screening for test substances for a therapeutic and/or prophylactic drug for acute myeloid leukemia or myelodysplastic syndrome (MDS) by using each index.

In one embodiment of the present invention using the number of hematopoietic progenitor cells as an index, a therapeutic or prophylactic drug for acute myeloid leukemia or myelodysplastic syndrome (MDS) can be screened for by a method including the following steps:
(a) a step of contacting hematopoietic progenitor cells induced from iPS cells produced from non-T cells in blood mononuclear cells isolated from a myelodysplastic syndrome patient (non-T cell-derived hematopoietic progenitor cells) and hematopoietic progenitor cells induced from control iPS cells (control hematopoietic progenitor cells) with a test substance, and
(b) a step of selecting the test substance as a candidate for a therapeutic or prophylactic drug for acute myeloid leukemia or myelodysplastic syndrome when the number of non-T cell-derived hematopoietic progenitor cells after contact with the test substance decreases from the number of control hematopoietic progenitor cells after contact with the test substance.

The iPS cells to be used here, which have been produced from non-T cells in blood mononuclear cells isolated from a myelodysplastic syndrome patient, are preferably derived from somatic cells of a myelodysplastic syndrome patient having chromosome abnormality. As used herein, the chromosome abnormality refers to the chromosome abnormality in the aforementioned myelodysplastic syndrome, which can be preferably at least one chromosome abnormality selected from the group consisting of increase in the copy number of short arm of chromosome 9 (9p), decrease in the copy number of short arm of chromosome 18 (18p), decrease in the copy number of long arm of chromosome 20 (20q), decrease in the copy number of long arm of chromosome 5 (5q), decrease in the copy number of long arm of chromosome 7 (7q), decrease in the copy number of long arm of chromosome 11 (11q), and translocation between long arm of chromosome 3 and long arm of chromosome 21.

The non-T cell-derived hematopoietic progenitor cells used here show lower colony-forming ability in a colony formation assay, as compared to hematopoietic progenitor cells prepared by inducing differentiation of normal MDS-iPS cells produced from somatic cells of a myelodysplastic syndrome patient. Examples of the colony showing low colony-forming ability include, but are not limited to, granulocyte macrophage colony (GM), macrophage colony (G) and granulocyte colony (M).

As the control iPS cells to be used here, iPS cells prepared from normal somatic cells collected from a myelodysplastic syndrome patient used for the production of iPS cells having the chromosome abnormality unique to myelodysplastic syndrome are desirably used to arrange other conditions. More preferably, iPS cells produced from T cells of a myelodysplastic syndrome patient are used.

In this screening method, more preferably, changes in the number of control hematopoietic progenitor cells are measured before and after contact with a test substance, and a test substance can be selected using suppression of decrease in the number of control hematopoietic progenitor cells, desirably, substantial absence of decrease in the number of control hematopoiesis cells, as a further index. Combined use of the thus-selected medicament with the below-mentioned therapeutic agent containing, as an active ingredient, hematopoietic progenitor cells induced to differentiate from iPS cells derived from T cells of a myelodysplastic syndrome patient results in the suppression of amplification of abnormal hematopoiesis cells without substantially preventing amplification of normal hematopoiesis cells, thereby enabling a more effective treatment.

As one embodiment of the method of screening for a therapeutic or prophylactic drug for acute myeloid leukemia or myelodysplastic syndrome of the present invention, a method including the following steps can be recited as example:
(a) a step of forming colonies of hematopoietic progenitor cells induced from iPS cells produced from non-T cells in blood mononuclear cells isolated from a myelodysplastic syndrome patient, in the presence of a test substance and in the absence of the test substance, and
(b) a step of selecting the test substance as a candidate for a therapeutic or prophylactic drug for acute myeloid leukemia or myelodysplastic syndrome when the colony number in the presence of the test substance increases from the colony number in the absence of the test substance.

In the present invention, “forming colonies” means separating hematopoietic progenitor cells, and culturing them in a semi-solid medium such as methylcellulose, soft agar and the like in the presence of cytokine to form a cell population (i.e., colony). Cytokine used for colony formation is, for example, one or more selected from the group consisting of SCF, G-CSF, GM-CSF, IL-3, IL-6 and EPO. A colony can be formed using a commercially available kit and examples thereof include CytoSelect of Cell Biolabs and the like.

In the present invention, the colony number may be counted by visual observation or instrumentally counted using IN Cell Analyzer. In addition, the obtained colonies may be dissolved, and counted using Cyquant GR Dye of Cell Biolabs.

As one embodiment of the method of screening for a therapeutic or prophylactic drug for acute myeloid leukemia or myelodysplastic syndrome of the present invention, a method including the following steps can be recited as example:
(a) a step of inducing iPS cells produced from non-T cells in blood mononuclear cells isolated from a myelodysplastic syndrome patient into hematopoietic progenitor cells,
(b) a step of inducing the aforementioned hematopoietic progenitor cells into hematopoietic cells in the presence of a test substance, and in the absence of the test substance, and
(c) a step of selecting the test substance as a candidate for a therapeutic or prophylactic drug for acute myeloid leukemia or myelodysplastic syndrome when the number of hematopoietic cells in the presence of the test substance increases from the number of hematopoietic cells in the absence of the test substance.

In the present invention, the hematopoietic cell is selected from the group consisting of neutrophil, eosinophil, basophil, red blood cell and megakaryocyte.

When the hematopoietic cells are red blood cells in the present invention, a therapeutic or prophylactic drug for acute myeloid leukemia or myelodysplastic syndrome (MDS) can be screened for by a method including the following steps:
(a) a step of inducing iPS cells produced from non-T cells in blood mononuclear cells isolated from a myelodysplastic syndrome patient into hematopoietic progenitor cells,
(b) a step of inducing the aforementioned hematopoietic progenitor cells into red blood cells in the presence of a test substance, and in the absence of the test substance, and
(c) a step of selecting the test substance as a candidate for a therapeutic or prophylactic drug for acute myeloid leukemia or myelodysplastic syndrome when the number of red blood cells in the presence of the test substance increases from the number of hematopoietic cells in the absence of the test substance.

When the hematopoietic cells are neutrophils in the present invention, a therapeutic or prophylactic drug for acute myeloid leukemia or myelodysplastic syndrome (MDS) can be screened for by a method including the following steps:
(a) a step of inducing iPS cells produced from non-T cells in blood mononuclear cells isolated from a myelodysplastic syndrome patient into hematopoietic progenitor cells,
(b) a step of inducing the aforementioned hematopoietic progenitor cells into neutrophils in the presence of a test substance, and in the absence of the test substance, and
(c) a step of selecting the test substance as a candidate for a therapeutic or prophylactic drug for acute myeloid leukemia or myelodysplastic syndrome when the number of neutrophils in the presence of the test substance increases from the number of neutrophils in the absence of the aforementioned test substance.

When the hematopoietic cells are megakaryocytes in the present invention, a therapeutic or prophylactic drug for acute myeloid leukemia or myelodysplastic syndrome (MDS) can be screened for by a method including the following steps:
(a) a step of inducing iPS cells produced from non-T cells in blood mononuclear cells isolated from a myelodysplastic syndrome patient into hematopoietic progenitor cells,
(b) a step of inducing the aforementioned hematopoietic progenitor cells into megakaryocytes in the presence of a test substance, and in the absence of the test substance, and
(c) a step of selecting the aforementioned test substance as a candidate for a therapeutic or prophylactic drug for acute myeloid leukemia or myelodysplastic syndrome when the number of megakaryocytes in the presence of the test substance increases from the number of megakaryocytes in the absence of the test substance.

In the screening method of the present invention, any test substance can be used, which may be any known compound or novel compound and examples thereof include cell extract, cell culture supernatant, microorganism fermentation product, extract derived from marine organism, plant extract, purified protein or crude protein, peptide, non-peptidic compound, synthetic low-molecular compound, natural compound and the like. In the present invention, a test substance can also be obtained using any of many approaches in combinatorial library methods known in the pertinent technical field including (1) biological library method, (2) synthetic library method using deconvolution, (3) “one-bead one-compound” library method, and (4) synthetic library method using affinity chromatography selection. While a biological library method using affinity chromatography selection is limited to peptide library, other four approaches can be applied to peptide, non-peptidic oligomer, and low-molecular compound library of a compound (Lam (1997) Anticancer Drug Des. 12: 145-67). Examples of the synthesis method of a molecule library can be found in the pertinent technical field (DeWitt et al. (1993) Proc. Natl. Acad. Sci. USA 90: 6909-13; Erb et al. (1994) Proc. Natl. Acad. Sci. USA 91: 11422-6; Zuckermann et al. (1994) J. Med. Chem. 37: 2678-85; Cho et al. (1993) Science 261: 1303-5; Carell et al. (1994) Angew. Chem. Int. Ed. Engl. 33: 2059; Carell et al. (1994) Angew. Chem. Int. Ed. Engl. 33: 2061; Gallop et al. (1994) J. Med. Chem. 37: 1233-51). A compound library can be prepared as a solution (see Houghten (1992) Bio/Techniques 13: 412-21), bead (Lam (1991) Nature 354: 82-4), chip (Fodor (1993) Nature 364: 555-6), bacterium (U.S. Pat. No. 5,223,409), spore (U.S. Pat. Nos. 5,571,698, 5,403,484 and 5,223,409), plasmid (Cull et al. (1992) Proc. Natl. Acad. Sci. USA 89: 1865-9) or phage (Scott and Smith (1990) Science 249: 386-90; Devlin (1990) Science 249: 404-6; Cwirla et al. (1990) Proc. Natl. Acad. Sci. USA 87: 6378-82; Felici (1991) J. Mol. Biol. 222: 301-10; US-B-2002/103360).

The target diseases of the medicament, which are selected in the screening method of the present invention, are not limited to acute myeloid leukemia and myelodysplastic syndrome (MDS) only, and examples thereof include, but are not limited to, diseases accompanied by a decrease in the number of red blood cells, diseases accompanied by a decrease in the number of neutrophils, diseases accompanied by a decrease in the number of hematopoietic progenitor cells and/or lowering of the hematopoietic function and the like. To be specific, the target diseases of the medicament, which are selected in the screening method of the present invention, include congenital anemia, aplastic anemia, autoimmune anemia, myelodysplastic syndrome (MDS), agranulocytosis, lymphocyte reducing symptom, thrombocytopenia, hematopoietic stem cell and/or hematopoietic progenitor cell reducing symptoms associated with various carcinomas or tumors, hematopoietic stem cell and/or hematopoietic progenitor cell reducing symptoms associated with cancer chemotherapy or radiation therapy, acute radioactive syndrome, delayed recovery of hematopoietic stem cell and/or hematopoietic progenitor cell after transplantation of bone marrow, cord blood or peripheral blood, hematopoietic stem cell and/or hematopoietic progenitor cell reducing symptoms associated with transfusion, leukemia (including acute myeloid leukemia (AML), acute lymphoid leukemia (ALL), chronic myeloid leukemia (CML), chronic lymphoid leukemia (CLL)), malignant lymphoma, multiple myeloma, myeloproliferative disease, hereditary blood diseases and the like. More suitable target diseases of the medicament, which are selected in the screening method of the present invention, can be acute myeloid leukemia and myelodysplastic syndrome as mentioned above.

### Therapeutic Agent for Myelodysplastic Syndrome (MDS)

The present invention provides a therapeutic agent for myelodysplastic syndrome, comprising hematopoietic progenitor cells prepared by differentiation induction of iPS cells produced from somatic cells of a myelodysplastic syndrome patient. The somatic cells of a myelodysplastic syndrome patient to be used in the present invention are preferably cells derived from the patient, and are free of chromosome abnormality. While the somatic cells of a myelodysplastic syndrome patient to be used in the present invention are, for example, T cells, the cells are not limited thereto.

Hematopoietic progenitor cells contained in the therapeutic agent for myelodysplastic syndrome of the present invention may be derived from a patient to be the subject of the treatment, or may be derived from other myelodysplastic syndrome patient. Preferably, it is derived from a patient to be the subject of the treatment. When the hematopoietic progenitor cell is derived from other myelodysplastic syndrome patient, somatic cells are preferably collected from other person having the same HLA type to prevent occurrence of the rejection.

The medicament of the present invention may contain induced hematopoietic progenitor cells singly or buffer, antibiotic, other medicament additives and the like may be contained together with the hematopoietic progenitor cells.

The medicament of the present invention is not limited to acute myeloid leukemia and myelodysplastic syndrome (MDS) only, and is also effective as a therapeutic agent for, for example, diseases accompanied by a decrease in the number of red blood cells, diseases accompanied by a decrease in the number of neutrophils, diseases accompanied by a decrease in the number of hematopoietic progenitor cells and/or lowering of the hematopoietic function and the like. To be specific, the target diseases of the therapeutic agent of the present invention include congenital anemia, aplastic anemia, autoimmune anemia, myelodysplastic syndrome (MDS), agranulocytosis, lymphocyte reducing symptom, thrombocytopenia, hematopoietic stem cell and/or hematopoietic progenitor cell reducing symptoms associated with various carcinomas or tumors, hematopoietic stem cell and/or hematopoietic progenitor cell reducing symptoms associated with cancer chemotherapy or radiation therapy, acute radioactive syndrome, delayed recovery of hematopoietic stem cell and/or hematopoietic progenitor cell after transplantation of bone marrow, cord blood or peripheral blood, hematopoietic stem cell and/or hematopoietic progenitor cell reducing symptoms associated with transfusion, leukemia (including acute myeloid leukemia (AML), acute lymphoid leukemia (ALL), chronic myeloid leukemia (CML), chronic lymphoid leukemia (CLL)), malignant lymphoma, multiple myeloma, myeloproliferative disease, hereditary blood diseases and the like. The diseases preferable for the application of the therapeutic agent of the present invention can be acute myeloid leukemia and myelodysplastic syndrome as mentioned above.

In the present invention, the route of administration of a medicament containing the hematopoietic progenitor cells to a patient is not particularly limited. For example, intravenous, subcutaneous, intradermal, intramuscular, intraperitoneal, intraosseous, and intracerebral administrations can be recited. A preferable administration route can be intravenous or intraosseous administration.

The hematopoietic progenitor cells of the present invention can also be used for a transplantation treatment, in which case a method similar to the conventional bone marrow transplantation and cord blood transplantation can be performed.

The dose of the medicament of the present invention to a patient varies depending on the kind of pathology to be treated, symptoms and severity of the disease, age, sex and body weight of the patient, administration method and the like and cannot be determined unambiguously. However, a suitable dose can be determined as appropriate based on the judgment of the doctor in consideration of the aforementioned situation.

While the present invention is further specifically explained by the following Examples, the scope of the present invention is not limited to the Examples.

### EXAMPLE 1

### Establishment of iPSCs Derived from MDS Patients

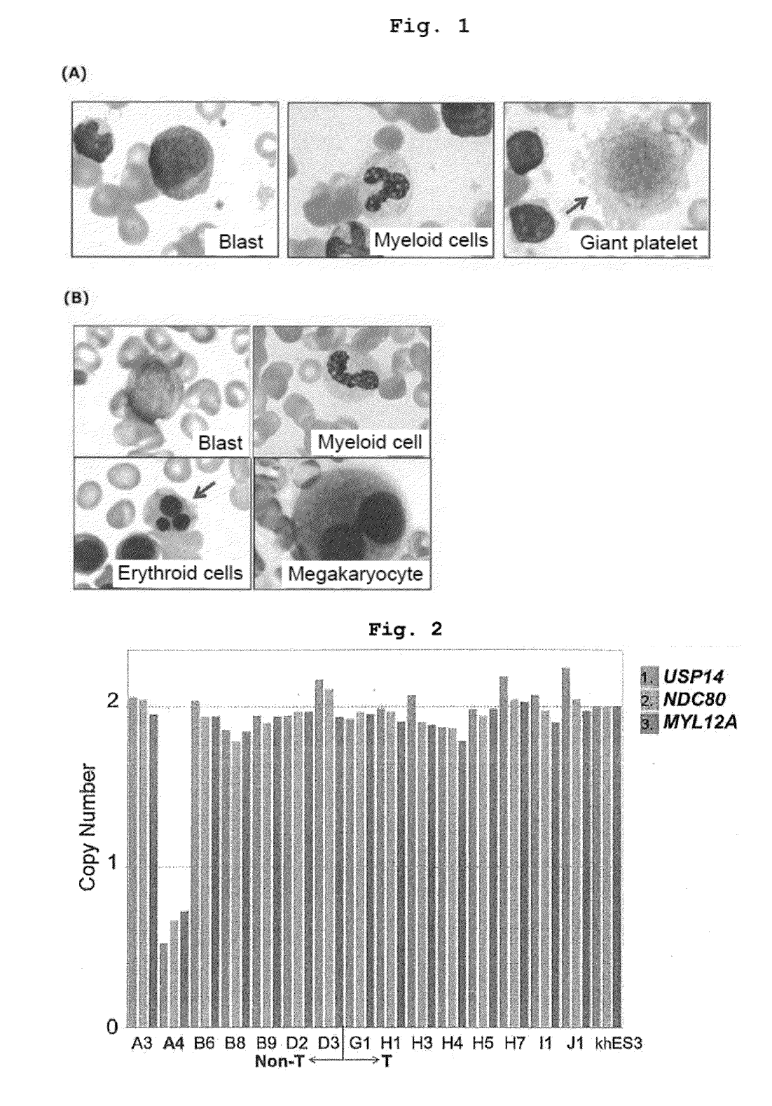

According to the method described in Okita. K, et al., Stem Cells. 2012 Nov. 29., iPS cells were prepared from the peripheral blood of 4 myelodysplastic syndrome (MDS) patients (KM3, KM5, KM15 and KM16; stained images of the blood derived from KM3 and KM5 are shown in FIG. 1) who were confirmed to show emergence of blasts, and images of blood cell dysplasia in bone marrow and peripheral blood. The detail is as follows. In Kyoto University Hospital, blood samples were collected from these MDS patients after obtaining informed consent, peripheral blood mononuclear cells (PMNC) were recovered from the collected blood by a density gradient centrifugation method using Ficoll-paque Plus (GE Healthcare) or BD Vacutainer CPT (BD). Using Nucleofector 2b Device (Lonza) and Amaxa(R) Human T Cell Nucleofector (R) Kit, 3 μg of an expression plasmid mixture was introduced into 3 to 5×10⁶ cells of PMNC. The expression plasmid used then was a combination of pCXLE-hOCT3/4-shp53-F, pCXLE-hSK and pCXLE-hUL, and pCXWB-EBNA1. The cells (3×10⁴ to 1×10⁶ cells) introduced with the expression plasmid were transferred to a 6-well culture plate (Falcon) seeded with MEF feeder cells (mitomycin C-treated) in advance, and cultured under the conditions of 37° C., 5% CO₂. To establish iPS cells derived from T cells, X-vivo10 medium (Lonza) added with 30 U/ml IL-2 (PeproTech) and 5 μl/well Dynabeads Human T-activator CD3/CD28 was used. On the other hand, to establish iPS cells derived from blood cells (non-T cells) other than T cells, αMEM medium added with 10% FCS, 10 ng/ml IL-3, 10 ng/ml IL-6, 10 ng/ml G-CSF and 10 ng/ml GM-CSF was used. On day 2 of plasmid introduction, an equal amount of medium for primate ES cells (REPROCELL) added with bFGF and 10 μM Y27632 was added to each well. Then, on day 4 of plasmid introduction, the culture medium was exchanged with a medium for primate ES cells (REPROCELL) added with bFGF and 10 μM Y27632. On days 20-25 of plasmid introduction, ES-like colonies (iPS cell colonies) were picked up. In this way, iPS cells derived from T cells and iPS cells derived from non-T cells were prepared.

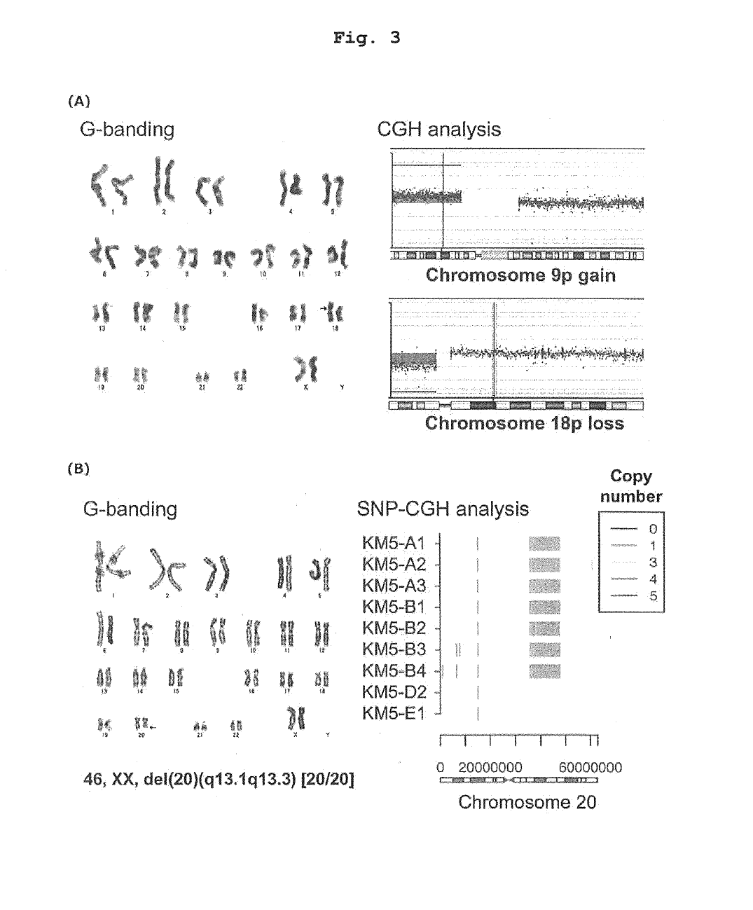

Sequentially, the copy number of USP14, NDC80 and MYL12A of each iPS cell line prepared from the blood derived from an MDS patient (KM3) was measured. As a result, it was confirmed that the copy number of A4 line (KM3-A4: iPS cells derived from non-T cells) was not more than half (FIG. 2). Furthermore, the CGH (Comparative genomic hybridization) array analysis of the same KM3-A4 line was performed. As a result, it was confirmed that the copy number of short arm of chromosome 9 (9p) increased and the copy number of short arm of chromosome 18 (18p) decreased (FIG. 3A, right figure). Similar abnormality was confirmed in the karyotype analysis at that time by the G band method (FIG. 3A, left figure). On the other hand, none of the iPS cell lines prepared from the T cells of the same MDS patient (KM3) (G1, H1, H3, H4, H5, H7, I1, J1) showed chromosome abnormality (FIG. 2).

From the above, it was confirmed that KM3-A4 line establish from non-T cells of an MDS patient (KM3) were iPS cells derived from the cells having chromosomal abnormality, and all iPS cells derived from T cells were iPS cells having normal chromosome even though they were derived from an MDS patient. This means that iPS cells derived from T cells of the same patient can be used as a normal control.

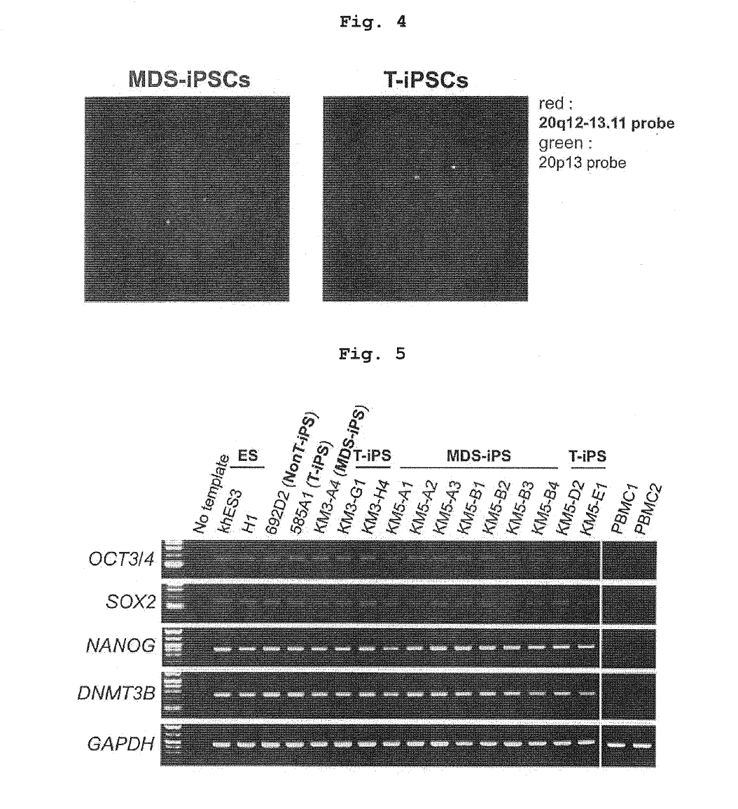

Similarly, SNP-CGH array analysis of 9 lines of iPS cells established from the blood cells derived from an MDS patient (KM5) was performed. As a result, it was confirmed that the copy number of long arm of chromosome 20 (20q) decreased in A1, A2, A3, B1, B2, B3 and B4 lines (KM5-A1, KM5-A2, KM5-A3, KM5-B1, KM5-B2, KM5-B3 and KM5-B4, respectively) (FIG. 3B, right figure). Similar abnormality was also confirmed in the karyotype analysis at that time by the G band method (FIG. 3B, left figure). It was also confirmed in KM5 that iPS cells to be the normal control can be obtained from the same patient (FIG. 3B, right figure, KM5-D2, KM5-E1). Furthermore, the number of the 20q region was confirmed by the FISH method to confirm only one pair of 20q12-13 in mutant iPS cells (FIG. 4).

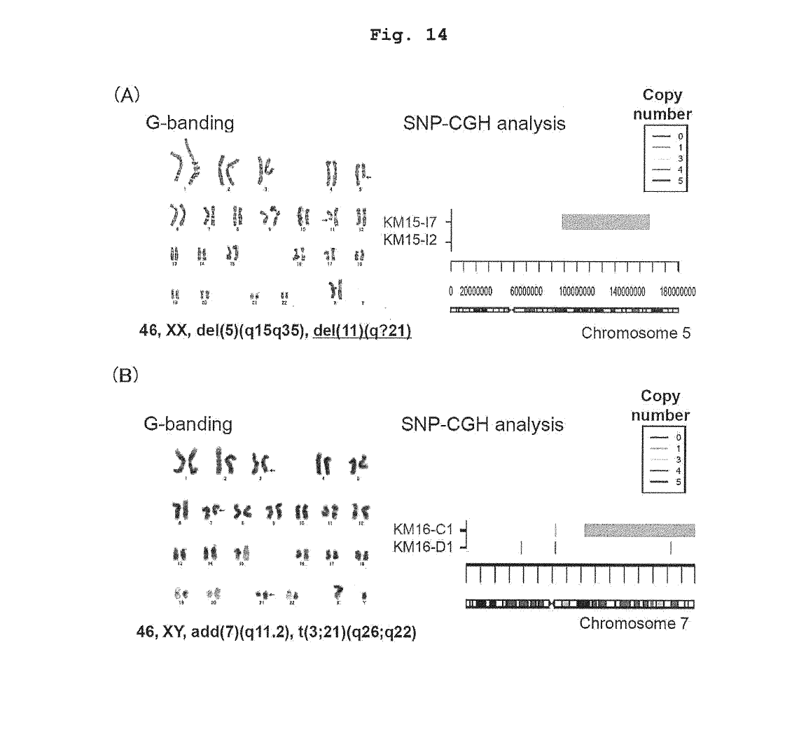

Similarly, SNP-CGH array analysis of iPS cells established from the blood cells derived from an MDS patient (KM15) was performed. As a result, it was confirmed that the copy number of long arm of chromosome 5 (5q) and long arm of chromosome 11 (11q) decreased in 17 line (KM15-17) (FIG. 14A). Similar abnormality was also confirmed in the karyotype analysis at that time by the G band method (FIG. 14A, left figure). It was also confirmed in KM15 that iPS cells to be the normal control can be obtained from the same patient (FIG. 14A, right figure, KM15-I2).

Similarly, SNP-CGH array analysis of iPS cells established from the blood cells derived from an MDS patient (KM16) was performed. As a result, it was confirmed that the copy number of long arm of chromosome 7 (7q) decreased and long arm of chromosome 3 and long arm of chromosome 21 were translocated in C1 line (KM16-C1) (FIG. 14B). Similar abnormality was also confirmed in the karyotype analysis at that time by the G band method (FIG. 14B, left figure). It was also confirmed in KM16 that iPS cells to be the normal control can be obtained from the same patient (FIG. 14B, right figure, KM16-D1).

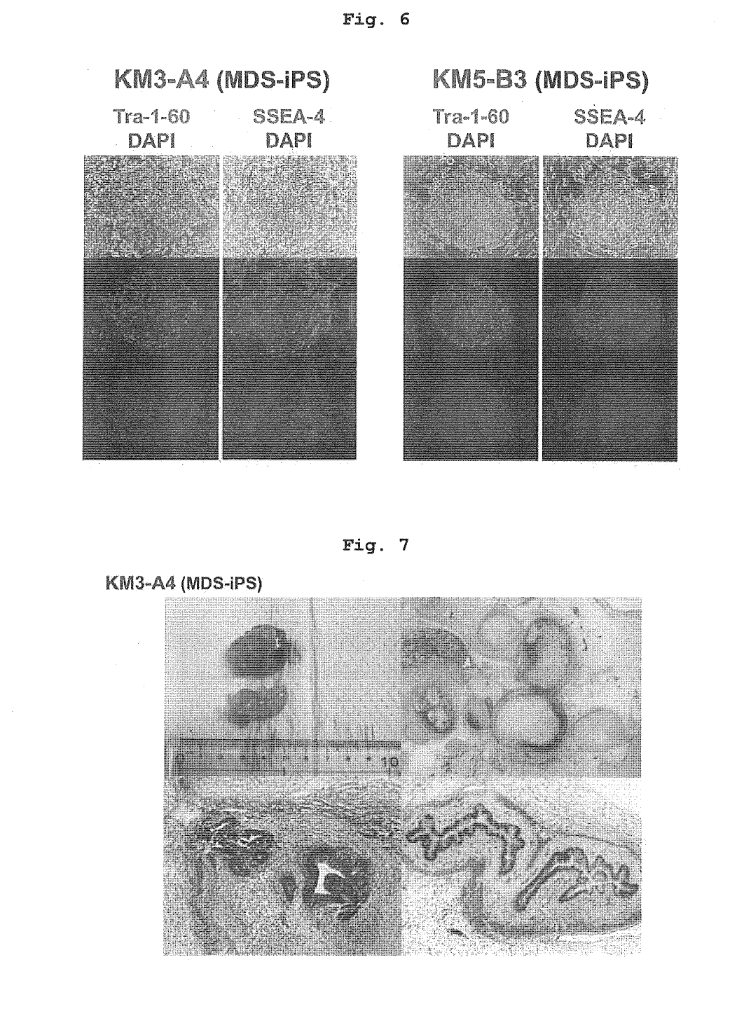

The pluripotency marker of iPS cells derived from the obtained KM3 and KM5 was confirmed by RT-PCR. As a result, it was confirmed that a pluripotency marker was expressed (FIG. 5) as in existing ES cells (KhES3 and H1) and iPS cells (692D2 and 585A1). It was also confirmed that a surface marker of pluripotency was similarly expressed (FIG. 6). Furthermore, the presence of teratoma forming ability was confirmed (FIG. 7). From the above, it was confirmed that mutant iPS cells (MDS-iPSCs) and normal iPS cells (Normal iPSCs and T-iPSCs) are not different in the function of pluripotency.

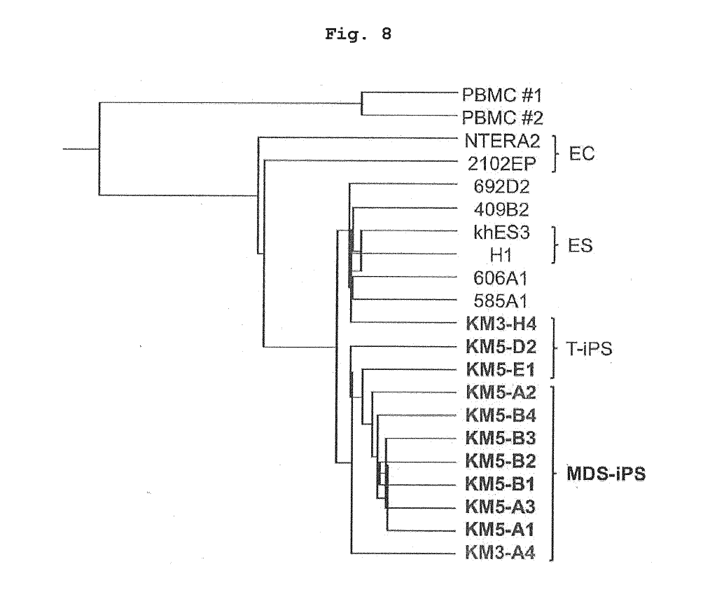

On the other hand, the gene expression of ES cells, existing iPS cells and iPS cells derived from MDS patients was analyzed by the microarray method and hierarchical clustering was performed. As a result, it was confirmed that mutant iPS cells tend to show gene expression profile different from that of normal iPS cells even in iPS cells derived from the same patient (FIG. 8).

### EXAMPLE 2

### Differentiation Induction into Hematopoietic Progenitor Cell

To differentiate iPS cells (MDS-iPSCs and Normal iPSCs) into hematopoietic progenitor cells (HPCs), iPS cells were cultured using each of OP9 stromal cell coculture system and EB method.

In the OP9 stromal cell coculture system, iPS cell clusters (<100 cells) were seeded using 10 m of HPC differentiation medium (α-MEM supplemented with 10% FBS, 5.5 mg/ml human transferrin, 2 mM L-glutamine, 0.5 mM α-monothioglycerol, 50 μg/mL ascorbic acid, and 20 ng/ml vascular endothelial growth factor (VEGF)) in a 10 cm dish coated with gelatin in advance and containing OP9 cultured to overconfluence. The next day, the medium was exchanged with 20 ml of fresh HPC differentiation medium, and the medium was exchanged with HPC differentiation medium every 3 days. On days 12-14, the colonies were treated with 5 ml of collagenase Type IV (1 mg/ml) for 30 min, and dissociated using 0.05% Trypsin-EDTA at 37° C. for 20 min. To remove stromal cells, 5-fold amount of OP9 medium was added to the dissociated cells to allow for re-suspending, cultured on a culture vessel at 37° C. for 45 min and floating cells were recovered. Furthermore, to remove stromal cells and aggregated cells, recovered cells were passed through a 100 μm filter and the differentiation induced cells were evaluated by FACS.

On the other hand, in the EB method, the cells were induced to differentiate into hematopoietic progenitor cells by the following procedures.
(day 0-day 1) Using a 6-well low-attachment plate, and in an environment of 37° C., 5% CO₂, 5% O₂ and 90% N₂, clusters (10-20 cells) of iPS cells free of feeder cells were cultured for 24 hr in a aggregation medium composed of StemPro-34 supplemented with 10 ng/mL human bone morphogenetic protein-4 (BMP-4), 2 mM glutamine, penicillin/streptomycin, 0.4 mM α-monothioglycerol and 50 μg/mL ascorbic acid, whereby embryoid bodies (EBs) were prepared.
(day 1-day 4) The obtained EBs were recovered, washed, and further cultured for 3 days in StemPro-34 supplemented with 5 ng/mL bFGF, 10 ng/mL BMP-4, 2 mM glutamine, penicillin/streptomycin, 0.4 mM α-monothioglycerol and 50 μg/mL ascorbic acid, whereby primitive streak/mesoderm formation was induced.
(day 4-day 8) On day 4, EBs were recovered again, and cultured again for 4 days in StemPro-34 supplemented with vascular endothelial growth factor (VEGF; 10 ng/mL), bFGF (1 ng/mL), interleukin-6 (IL-6; 10 ng/mL), IL-3 (40 ng/mL), IL-11 (5 ng/mL), stem cell factor (SCF; 100 ng/mL) and human FLT3 ligand (FLT3L; 100 ng/ml) for the specification into hematopoietic progenitor cells and development thereof.
(day 8-day 15) On day 8, EBs were transferred to a 5% CO₂/air environment, placed in StemPro-34 supplemented with VEGF (10 ng/mL), erythropoietin (EPO; 4 U/mL), thrombopoietin (TPO; 50 ng/mL), SCF, FLT3L, IL-6, IL-11 and IL-3, and further cultured for 7 days for the maturation to hematopoietic progenitor cells (maturation to erythroblasts and megakaryoblastic progenitor cells) and growth. The cells were incubated in 0.25% Trypsin-EDTA at 37° C. for 5-10 min, and the cells were dissociated with 1000 μl pipette to give single cell suspension. Then, the dissociated cells were passed through a 70 μm filter and the differentiation induced cells were evaluated by FACS.

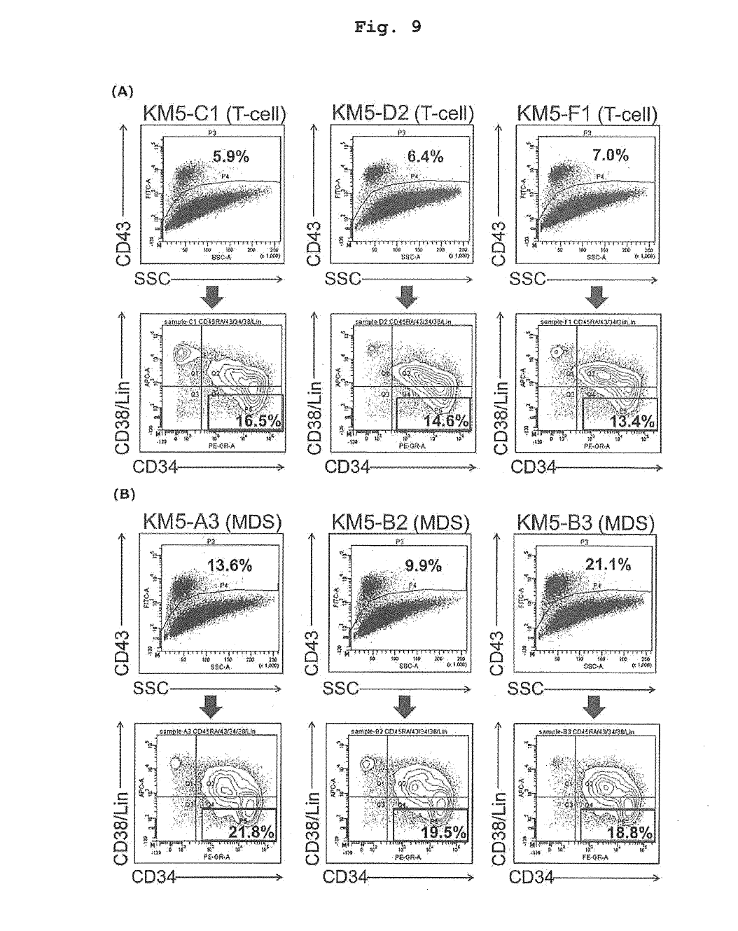

From the above results, the differentiation potency of MDS-iPSCs into hematopoietic progenitor cells was of the same level as Normal iPSCs by any of the methods of OP9 stromal cell coculture system and EB method (FIG. 9 and FIG. 11).

### EXAMPLE 3

### Colony Formation Assay of Hematopoietic Progenitor Cells Derived from MDS-iPSCs and Normal iPSCs

Using a 35 mm culture dish, 2500 cells from the cells of CD43⁺CD34⁺CD38⁻ fraction induced to differentiate from iPS cells (abnormal MDS-iPSCs (NonT-iPS) and Normal iPSCs (T-iPS)) and extracted by flow cytometer were seeded in 2 ml of a methylcellulose medium containing SCF, G-CSF, GM-CSF, IL-3, IL-6 and EPO (MethoCult H4435) according to the method of Example 2. After 15 days, the number of the colonies was counted under a microscope. After Cytospin, Wright's staining was performed, and the colony type was identified by microscopic observation.

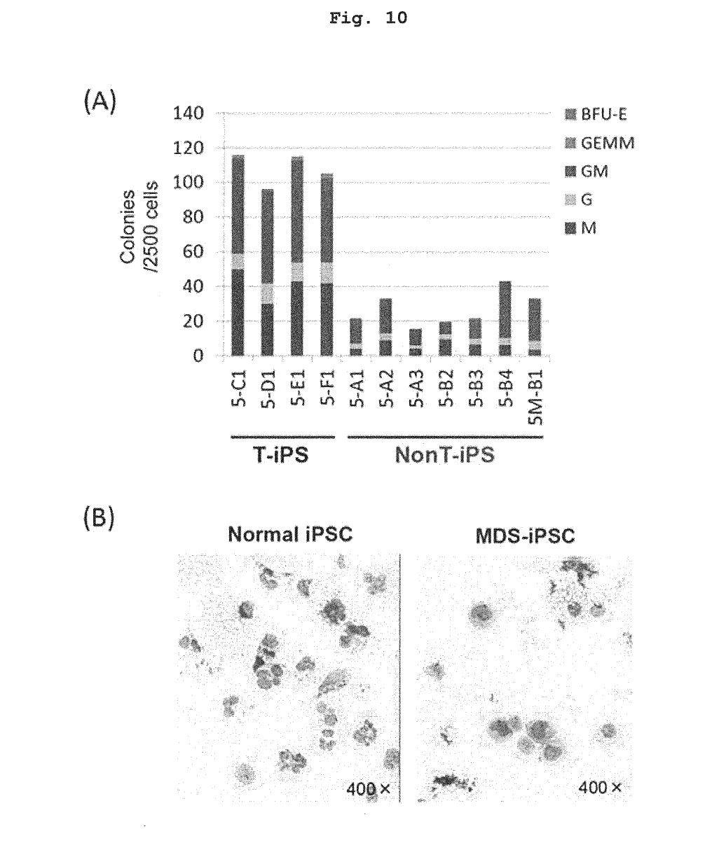

As a result, it was confirmed that hematopoietic progenitor cells derived from MDS-iPSCs have significantly low lo colony-forming ability as compared to Normal iPSCs (FIGS. 10 and 15). In addition, the ratio of neutrophils (segmented leukocytes and stab cells) in nucleated cells on day 15 of colony assay was 40% for hematopoietic progenitor cells derived from Normal iPSCs, and as low as 2% for hematopoietic progenitor cells derived from MDS-iPSC. Here, since 5M-B1 is MDS-iPSCs free of mutation in the karyotype, iPS cells derived from nonT cells are considered to have the characteristics of MDS independent of the mutation of the karyotype.

### EXAMPLE 4

### Differentiation Induction of MDS-iPSCs and Normal iPSCs into Red Blood Cells

Prior to differentiation induction, contamination with feeder cell was removed by culturing iPS cells (MDS-iPSCs (MDS-iPS) and Normal iPSCs (T-iPS)) in a mouse embryonic fibroblast conditioned medium supplemented with fibroblast growth factor (bFGF) on a plate coated with Matrigel for 72-96 hr.
(day 0-day 1) Using a 6-well low-attachment plate, and in an environment of 37° C., 5% O₂, 5% O₂ and 90% N₂, clusters (10-20 cells) of iPS cells free of feeder cells were cultured for 24 hr in a aggregation medium composed of StemPro-34 and supplemented with 10 ng/mL BMP-4, 2 mM glutamine, penicillin/streptomycin, 0.4 mM α-monothioglycerol and 50 μg/mL ascorbic acid, whereby embryoid bodies (EBs) were prepared. (day 1-day 4) The obtained EBs were recovered, washed, and further cultured for 3 days in StemPro-34 supplemented with 5 ng/mL bFGF, 10 ng/mL BMP-4, 2 mM glutamine, penicillin/streptomycin, 0.4 mM α-monothioglycerol and 50 μg/mL ascorbic acid, whereby primitive streak/mesoderm formation was induced.
(day 4-day 8) On day 4, EBs were recovered again, and cultured again for 4 days in StemPro-34 supplemented with VEGF (10 ng/mL), bFGF (1 ng/mL), IL-6 (10 ng/mL), IL-3 (40 ng/mL), IL-11 (5 ng/mL), SCF (100 ng/mL) and FLT3L (100 ng/ml) for the specification into hematopoietic progenitor cells and development thereof.
(day 8-day 18) On day 8, EBs were transferred to a 5% CO₂/air environment, placed in StemPro-34 supplemented with VEGF (10 ng/mL), EPO (4 U/mL), TPO (50 ng/mL), SCF, FLT3L (100 ng/ml), IL-6 (10 ng/mL), IL-11 (5 ng/mL) and IL-3 (40 ng/mL), and further cultured for 10 days for the maturation to hematopoietic progenitor cells (maturation to erythroblasts and megakaryoblastic progenitor cells) and growth. The cells were incubated in 0.25% Trypsin-EDTA at 37° C. for 5-10 min, and the cells were dissociated with 1000 μl pipette to give single cell suspension, whereby hematopoietic progenitor cells were obtained.
(day 18-day 26) The above-mentioned hematopoietic progenitor cells were seeded at a density of 10⁶ cell/mL, and cultured in StemPro-34 supplemented with SCF (100 ng/ml), IL-3 (5 ng/ml) and EPO (4 U/mL) for 8 days.
(day 26-day 33) The obtained cells were cultured in fresh StemPro-34 supplemented with SCF and EPO, and the ratio of CD235a-positive cells was determined by flow cytometry on days 30-33.

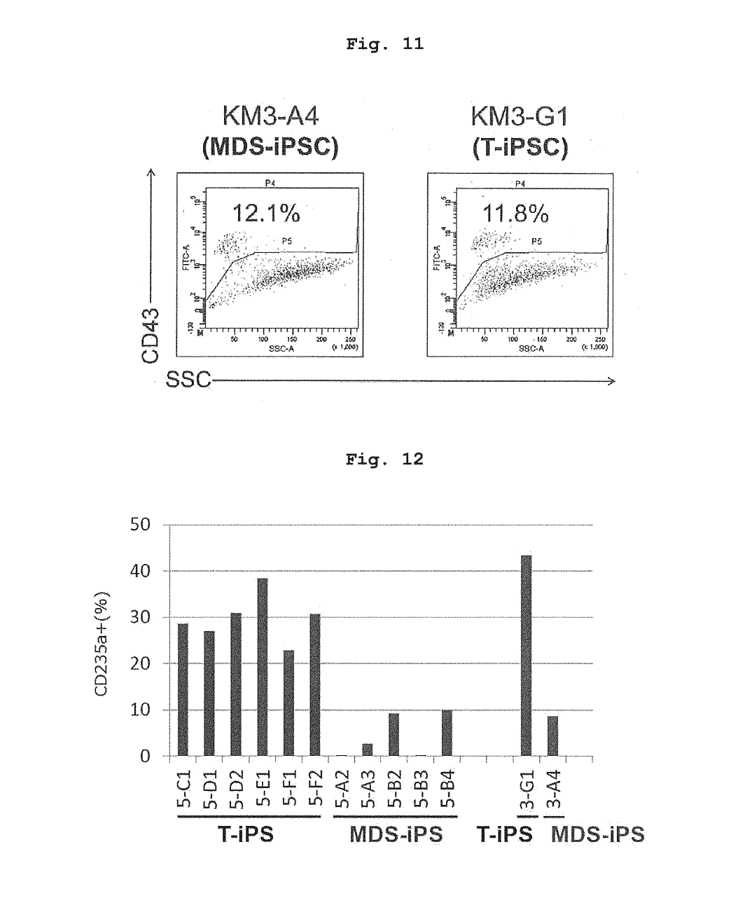

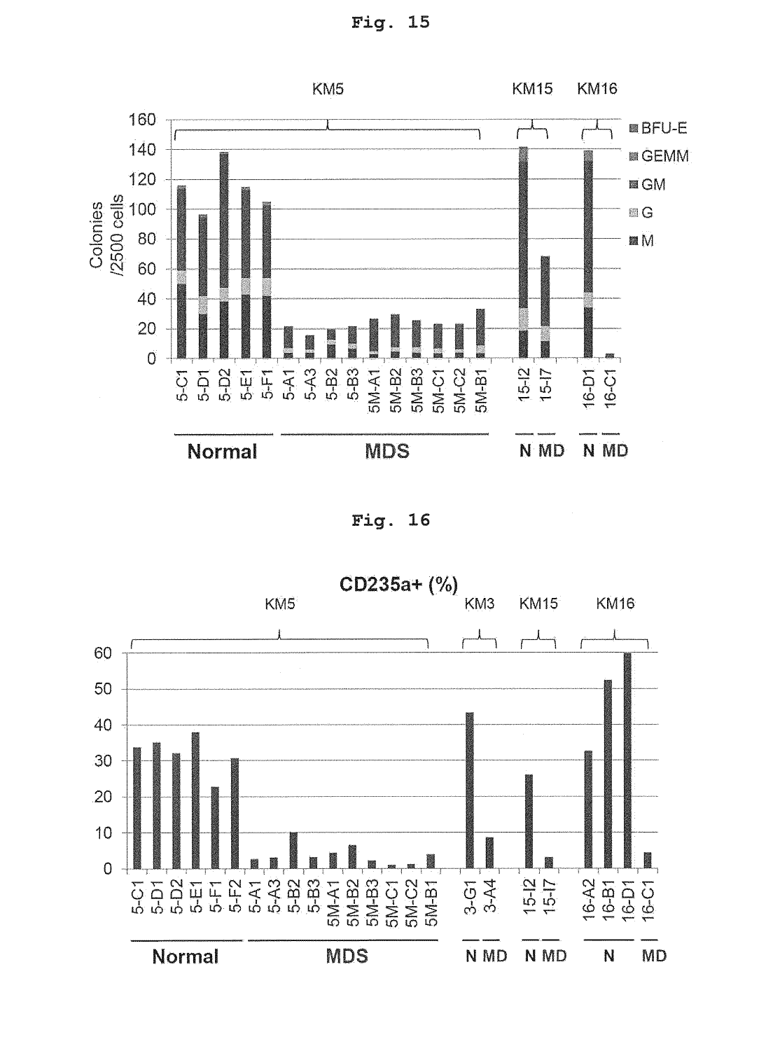

As a result, low differentiation inducibility into red blood cells was shown for MDS-iPSCs, as compared to Normal iPSCs (FIGS. 12 and 16).

### EXAMPLE 5

### Differentiation Induction of MDS-iPSCs and Normal iPSCs into Neutrophils

To differentiate iPS cells (abnormal MDS-iPSCs (NonT-iPS) and Normal iPSCs (T-iPS)) into hematopoietic progenitor cells (HPCs), iPS cells were cultured using OP9 stromal cell coculture system. In brief, clusters (<100 cells) of iPS cells were seeded in 10 cm dish coated with gelatin and containing OP9 cultured to overconfluence in advance using in 10 ml of HPC differentiation medium. The next day, the medium was exchanged lo with 20 ml of a fresh HPC differentiation medium and the HPC differentiation medium was exchanged every 3 days. On days 11-12, the colonies were treated with 5 ml of collagenase Type IV (1 mg/ml) for 30 min, and dissociated using 0.05% Trypsin-EDTA at 37° C. for 20 min. The dissociated cells were resuspended in a medium for proliferation of pluripotent bone marrow progenitor cells (α-MEM supplemented with 10% FBS, 5.5 mg/ml human transferrin, 2 mM L-glutamine, 0.5 mM α-monothioglycerol, 50 μg/ml ascorbic acid, and 20 ng/ml GM-CSF). Two days later, bone marrow culture was recovered, and the cells were passed through a 70 μm filter to remove aggregated cells. Lin-CD34⁺CD43⁺CD45⁺ precursor cells were extracted by flow cytometry, and seeded on a 6-well plate containing OP9 cultured to semiconfluence in 2.5 ml of a neutrophil differentiation medium (IMDM supplemented with 20% FBS and 100 ng/ml G-CSF). On day 3, 2.5 ml of a neutrophil differentiation medium was added and, on day 6, a half of the neutrophil differentiation medium was exchanged. After 9-10 days, the ratio of CD66b positive neutrophil cells in CD11b positive bone marrow cells was determined by flow cytometry.

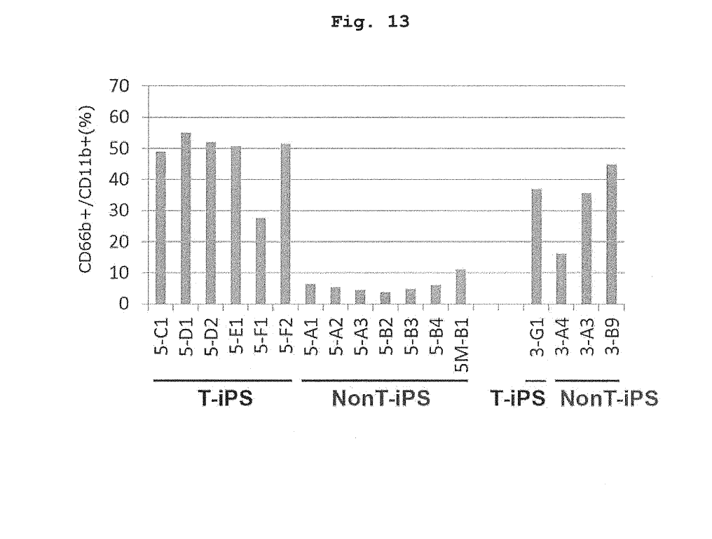

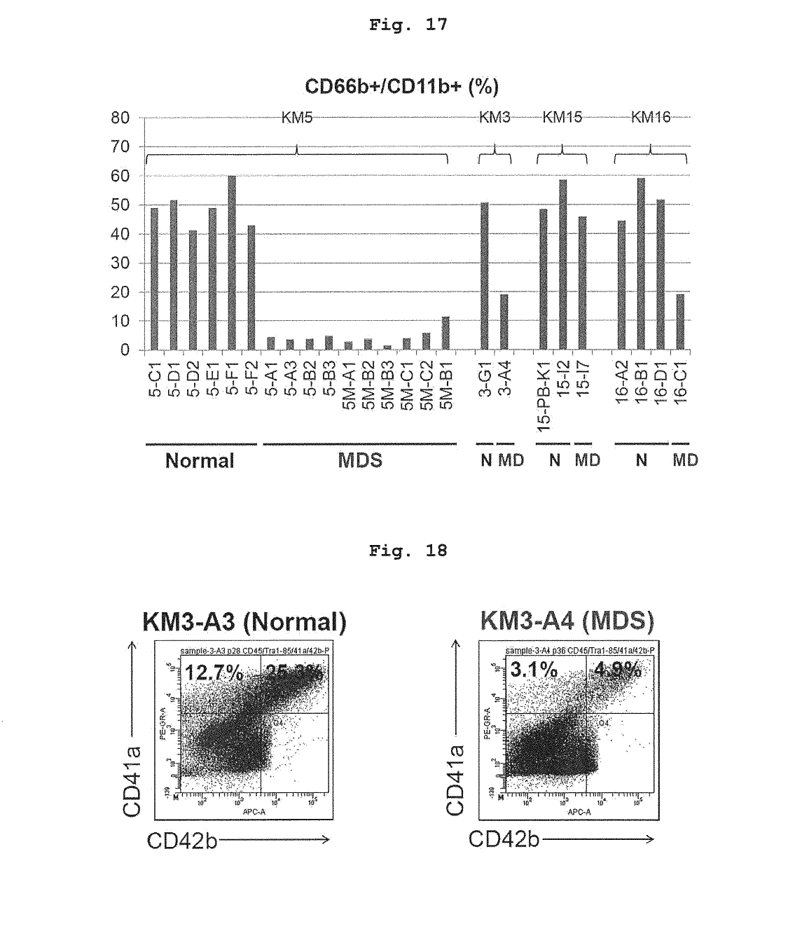

As a result, low differentiation inducibility into neutrophils was shown for MDS-iPSCs, as compared to Normal iPSCs (FIGS. 13 and 17).

### EXAMPLE 6

### Differentiation Induction of MDS-iPSCs and Normal iPSCs into Megakaryocytes

To differentiate iPS cells (MDS-iPSCs and Normal iPSCs) into hematopoietic progenitor cells (HPCs), iPS cells were cultured using the OP9 stromal cell coculture system described in Example 5. On days 12-14, the colonies were treated with 5 ml of collagenase Type IV (1 mg/ml) for 30 min, and dissociated using 0.05% Trypsin-EDTA at 37° C. for 20 min. To remove bone marrow interstitial cells, the dissociated cells were re-suspended in 5-fold amount of OP9 medium, seeded on a culture vessel, and incubated on a culture vessel at 37° C. for 45 min, and floating cells were recovered. Furthermore, to remove bone marrow interstitial cells and aggregated cells, the cells were passed through a 100 μm filter and seeded in a OP9 semiconfluent 6-well plate supplemented with 2 ml of megakaryocyte differentiation medium (α-MEM supplemented with 10% FBS, 5.5 mg/ml human transferrin, 2 mM L-glutamine, 0.5 mM α-monothioglycerol, 50 μg/ml ascorbic acid, 100 ng/ml TPO and 100 ng/ml SCF). Two days later, floating cells were recovered, and seeded in a new OP9 semiconfluent 6-well plate supplemented with 2 ml of megakaryocyte differentiation medium. On day 4, 2 ml of megakaryocyte differentiation medium was added. On days 6 and 8, half the medium was exchanged. On day 10, the ratio of CD41a/CD42b double positive cells in CD45 positive cells was determined by flow cytometry.

As a result, low differentiation inducibility into megakaryocytes was shown for MDS-iPSCs, as compared to Normal iPSCs (FIG. 18).

### EXAMPLE 7

### Improving Effect of p38 Inhibitor on Colony-Forming Ability

To study whether the decreased colony-forming ability of hematopoietic progenitor cells induced from MDS-iPS cells is recovered by the effect of p38 inhibitor, colony formation assay was performed by a method similar to Example 3 except that a p38 inhibitor was added. In brief, using a 35 mm culture dish, 2500 cells from the cells of CD43+CD34+CD38-fraction induced to differentiate from iPS cells (MDS-iPSCs (NonT-iPS)) and Normal iPSCs (T-iPS)) and extracted by flow cytometer were seeded in 1.1 ml of a methylcellulose medium containing SCF, G-CSF, GM-CSF, IL-3, IL-6, EPO and 10 μM SB203580 (p38 inhibitor) according to the method of Example 2. After 14 or 15 days, the number of colonies was counted under a microscope. Furthermore, after Cytospin, Wright's staining was performed, and the colony type was identified by microscopic observation.

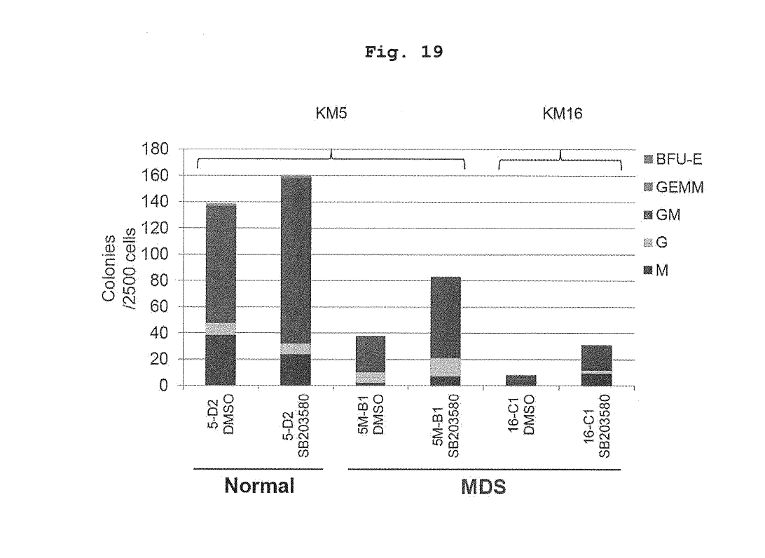

As a result, application of a p38 inhibitor showed an improving effect on the colony-forming ability (FIG. 19). This suggests that differentiated cells derived from iPS cells derived from MDS patients are useful for screening for a therapeutic drug for MDS and acute myeloid leukemia to which MDS transited.

### INDUSTRIAL APPLICABILITY

The present invention is based on the successful reproduction of pathology of myelodysplastic syndrome in the hematopoietic cells prepared by differentiation induction of iPS cells produced from somatic cells of myelodysplastic syndrome patients, particularly, hematopoietic progenitor cells, red blood cells and/or neutrophils. Therefore, a therapeutic or prophylactic drug for acute myeloid leukemia or myelodysplastic syndrome can be screened for by using the cells.

This application is based on a patent application No. 2013-109385 filed in Japan on May 23, 2013, the contents of which are incorporated by reference in full herein.

1. A method of screening for a therapeutic or prophylactic drug for acute myeloid leukemia or myelodysplastic syndrome, comprising the following steps:
(1) a step of providing hematopoietic progenitor cells induced from pluripotent stem (iPS) cells produced from non-T cells in blood mononuclear cells isolated from a myelodysplastic syndrome patient,
(2) a step of:
(a) culturing the hematopoietic progenitor cells to form colonies thereof in the presence of a test substance and in the absence of the test substance, or
(b) inducing differentiation of the hematopoietic progenitor cells into hematopoietic cells in the presence of a test substance and in the absence of the test substance, and
(3) a step of selecting the test substance as a candidate for a therapeutic or prophylactic drug for acute myeloid leukemia or myelodysplastic syndrome when the number of the colonies obtained in step (2)(a) or the hematopoietic cells obtained in step (2)(b) in the presence of the test substance is greater than that in the absence of the test substance.

2. The method according to claim 1, wherein the iPS cells produced from non-T cells in blood mononuclear cells isolated from the aforementioned myelodysplastic syndrome patient have at least one chromosome abnormality selected from the group consisting of increase in the copy number of short arm of chromosome 9 (9p), decrease in the copy number of short arm of chromosome 18 (18p), decrease in the copy number of long arm of chromosome 20 (20q), decrease in the copy number of long arm of chromosome 5 (5q), decrease in the copy number of long arm of chromosome 7 (7q), decrease in the copy number of long arm of chromosome 11 (11q), and translocation between long arm of chromosome 3 and long arm of chromosome 21.

3.-4. (canceled)

5. The method according to claim 1 comprising the aforementioned step (2) (b), wherein the hematopoietic cells are red blood cells.

6. The method according to claim 1 comprising the aforementioned step (2) (b), wherein the hematopoietic cells are neutrophils.

7. The method according to claim 1 comprising the aforementioned step (2) (b), wherein the hematopoietic cells are megakaryocytes.

8. The method according to claim 5, wherein the aforementioned step (2) (b) comprises:
(i) culturing hematopoietic progenitor cells in a medium containing VEGF, IL-6, IL-3, IL-11, SCF, FLT3L, erythropoietin (EPO) and thrombopoietin (TPO),
(ii) culturing the cells obtained in step (i) in a medium containing IL-3, SCF and EPO, and
(iii) culturing the cells obtained in step (ii) in a medium containing SCF and EPO.

9. The method according to claim 6, wherein the aforementioned step (2) (b) comprises culturing the hematopoietic progenitor cells in a medium containing GM-CSF and/or G-CSF.

10. The method according to claim 7, wherein the aforementioned step (2) (b) comprises culturing the hematopoietic progenitor cells in a medium containing TPO and SCF.

11. The method according to claim 1, wherein the hematopoietic progenitor cells are induced by:
(1) culturing iPS cells in a medium containing BMP-4 to form embryoid bodies,
(2) culturing the aforementioned embryoid bodies in a medium containing bFGF and BMP-4, and
(3) culturing the cells obtained in step (2) in a medium containing bFGF, VEGF, IL-6, IL-3, IL-11, stem cell factor (SCF) and FLT3L.

12. The method according to claim 1, wherein the aforementioned hematopoietic progenitor cells are induced by coculturing iPS cells with feeder cells.

13. The method according to claim 12, wherein the aforementioned feeder cell is OP9 cell line.

14. A therapeutic method for myelodysplastic syndrome in a patient, comprising administering to the patient an therapeutically effective amount of hematopoietic progenitor cells differentiated from iPS cells produced from T cells of the patient.

15.-17. (canceled)

18. A method of screening for a therapeutic or prophylactic drug for acute myeloid leukemia or myelodysplastic syndrome, comprising the following steps:
(1) step of contacting hematopoietic progenitor cells in the following (a) and (b), respectively, with a test substance:
(a) hematopoietic progenitor cells induced from iPS cells produced from non-T cells (non-T cell-derived iPS cells) in blood mononuclear cells isolated from a myelodysplastic syndrome patient, and
(b) hematopoietic progenitor cells induced from control iPS cells, and
(2) a step of selecting the test substance as a candidate for a therapeutic or prophylactic drug for acute myeloid leukemia or myelodysplastic syndrome when the number of the hematopoietic progenitor cells of (a) after contact with the test substance is less than the number of the hematopoietic progenitor cells of (b) after contact with the test substance.

19. The method according to claim 18, wherein the aforementioned control iPS cells are produced from T cells in blood mononuclear cells isolated from the same patient as in the aforementioned step (1) (a).

20. The method according to claim 18, wherein the aforementioned non-T cell-derived iPS cells have at least one chromosome abnormality selected from the group consisting of increase in the copy number of short arm of chromosome 9 (9p), decrease in the copy number of short arm of chromosome 18 (18p), decrease in the copy number of long arm of chromosome 20 (20q), decrease in the copy number of long arm of chromosome 5 (5q), decrease in the copy number of long arm of chromosome 7 (7q), decrease in the copy number of long arm of chromosome 11 (11q), and translocation between long arm of chromosome 3 and long arm of chromosome 21.

21. The method according to claim 18, wherein the aforementioned hematopoietic progenitor cells (a) or (b) are induced by:
(1) culturing (a) the non-T cell-derived iPS cells or (b) the control iPS cells in a medium containing BMP-4 to form embryoid bodies,
(2) culturing the aforementioned embryoid bodies in a medium containing bFGF and BMP-4, and
(3) culturing the cells obtained in step (2) in a medium containing bFGF, VEGF, IL-6, IL-3, IL-11, stem cell factor (SCF) and FLT3L.

22. The method according to claim 18, wherein the aforementioned hematopoietic progenitor cells (a) or (b) are induced by coculturing (a) the non-T cell-derived iPS cells or (b) the control iPS cells with feeder cells.

23. The method according to claim 22, wherein the aforementioned feeder cell is OP9 cell line.
